(19) United States
(12) Patent Application Publication
(10) Pub. No.: US 2025/0382580 A1
(43) Pub. Date: Dec. 18, 2025

(54) COMPOSITIONS AND METHODS FOR ACCELERATED PRODUCTION OF THYMIC CELLS FROM PLURIPOTENT STEM CELLS

(71) Applicant: The Regents of the University of Colorado, a body Corporate, Denver, CO (US)

(72) Inventors: Holger A. Russ, Denver, CO (US); Lucas H. Armitage, Denver, CO (US)

(21) Appl. No.: 19/176,932

(22) Filed: Apr. 11, 2025

Related U.S. Application Data
(63) Continuation of application No. PCT/US23/36144, filed on Oct. 27, 2023.
(60) Provisional application No. 63/420,410, filed on Oct. 28, 2022.

(51) Int. Cl. C12N 5/071; A61K 35/39

(52) U.S. Cl. CPC C12N 5/0678; A61K 35/39; C12N 2501/113; C12N 2501/115; C12N 2501/119; C12N 2501/155; C12N 2501/16; C12N 2501/385; C12N 2501/41; C12N 2501/415; C12N 2501/42; C12N 2501/91; C12N 2502/00; C12N 2506/45

(57) ABSTRACT

Embodiments of the instant disclosure relate to novel compositions and methods for generating thymic cells. In some embodiments, thymic cells can be differentiated from pluripotent stem cells (PSC), anterior primitive streak (APS) cells, definitive endoderm (DE) cells, anterior foregut endoderm (AFE) cells, pharyngeal endoderm (PE) cells, ventral pharyngeal endoderm (VPE) cells, and third pharyngeal pouch endoderm (TPPE) cells using the compositions and methods disclosed herein. In certain embodiments, thymic cells generated by composition, systems and methods disclosed herein can be used to treat a health condition.

### PRIORITY

This U.S. application is a continuation application of International Application No. PCT/US2023/036144, filed Oct. 27, 2023, which claims benefit of U.S. Provisional Application No. 63/420,410, filed Oct. 28, 2022, which are incorporated herein by reference in their entireties for all purposes.

### FIELD

Embodiments of the instant disclosure relate to novel compositions and methods for generating thymic cells (thymic progenitor cells (TEPs) and thymic epithelial cells (TECs)) from pluripotent stem cells. In some embodiments, thymic cells can be differentiated from pluripotent stem cells (PSC), anterior primitive streak (APS) cells, definitive endoderm (DE) cells, anterior foregut endoderm (AFE) cells, pharyngeal endoderm (PE) cells, ventral pharyngeal endoderm (VPE) cells, and third pharyngeal pouch endoderm (TPPE) cells using compositions and methods disclosed herein.

### BACKGROUND

The thymus, an essential organ of the immune system, is the main site of T lymphocyte production and adaptive immunity regulation. The thymus is most active during childhood and involutes around the time of adolescence, resulting in a severe reduction or absence of naive T cell output, often leading to a compromised immune system in the elderly and in other health conditions. Thymic involution can be accelerated by certain clinical treatments, for example, chemotherapy. Reconstitution of adaptive immunity through mass production of different T cell types is therefore a therapeutic need in immunocompromised populations and for health conditions in need of adaptive immune therapies. Although pluripotent stem cells are a source of T cells for a variety of clinical applications, current methods of T cell differentiation result in the generation of cells with aberrant phenotypes and/or immature T cells that are not useful for therapy. In addition, negative selection, the process of removing autoreactive T cells during thymic T cell education, is currently not feasible. Other methods for generating thymic cells known in the art are inefficient. Therefore, a need exists for improved methods of generating thymic cells from renewable, readily available cell sources for the development of immune cell therapies and replacement therapies.

### SUMMARY

Embodiments of the instant disclosure relate to novel compositions and methods for generating thymic cells in vitro with improved efficiency. In certain embodiments, compositions for generating thymic cells at various stages of differentiation are disclosed. In accordance with these embodiments, a composition including a cell culture media and further including, but not limited to, at least one Wingless-related integration sites (Wnt) activator, at least one bone morphogenetic protein (BMP) inhibitor, and at least one of an Activin A activator or an Activin A inhibitor are described. In some embodiments, compositions disclosed herein can include a media as presented herein that can further include at least one of mammalian pluripotent stem cells (PSC), anterior primitive streak (APS) cells, definitive endoderm (DE) cells or combinations thereof.

In certain embodiments and further to paragraph [0004] above, compositions disclosed herein can be a medium having a base medium with low protein concentrations of about 1.0% (w/v) or less. In some embodiments, compositions disclosed herein can have a protein enriched cell culture medium having a concentration of protein of at least 0.5% (w/v) up to 30% (w/v) protein concentration. In accordance with certain embodiments herein, compositions disclosed herein having a protein enriched cell culture medium can further include at least one of definitive endodermal (DE) cells; anterior foregut endoderm (AFE), ventral pharyngeal endoderm (VPE), pharyngeal endoderm (PE), and third pharyngeal pouch endoderm (TPPE) cells.

In certain embodiments and further to paragraphs [0004]-[0005] above, compositions disclosed herein can and include media in absence of protein. In other embodiments, compositions disclosed herein can include a protein-enriched cell medium. In other embodiments, the medium can include, but is not limited to, MEM alpha. In certain embodiments, the medium can include Minimum Essential Medium (MEM), Eagle's Medium, Dulbecco's Modified Eagle Medium (DMEM), Dulbecco's Modified Eagle Medium: Nutrient Mixture F-12 (DMEM/F12), F10 Nutrient Mixture, Ham's F10 Nutrient Mix, Ham's F12 Nutrient Mixture, Medium 199, RPMI, RPMI 1640, reduced serum medium, basal medium (BME), DMEM/F12 (1:1), Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E and the like, and combinations thereof. In accordance with these embodiments, the media (e.g., MEM alpha media) can further include ribonucleosides, deoxyribonucleosides, and/or L-glutamine. In certain embodiments, the medium (e.g., MEM alpha media) can include, but is not limited to one or more or phenol red, polyvinyl alcohol, methylcellulose, non-essential amino acids, lipid concentrates, insulin/transferrin/selenium/ethanolamine (ITSE, ITS-X), and/or penicillin-streptomycin. In certain embodiments, the medium (e.g., MEM alpha media) can include, but is not limited to insulin/transferrin/selenium/ethanolamine (ITSE) or insulin/transferrin/selenium (ITS), ITSX, or the like. In accordance with these embodiments, compositions disclosed herein can include a supplemented media in the absence of protein or can be protein-enriched culture media, (e.g., MEM alpha media) and can further include at least one population of cells, including at least a population of definitive endodermal (DE) cells; a population of anterior foregut endoderm (AFE) cells, a population of ventral pharyngeal endoderm (VPE) cells, a population of pharyngeal endoderm (PE) cells, and a population of third pharyngeal pouch endoderm (TPPE) cells. In accordance with these embodiments, the at least one population of cells further includes a protein enriched media and one or more agents indicated herein. In other embodiments, the at least one population of cells further includes a media with little to no protein and one or more agents indicated herein.

In some embodiments and in furtherance of paragraphs [0004]-[0006] above, compositions disclosed herein can include a medium (e.g., MEM alpha media) having at least one Activin A activator, where the at least one Activin A activator can include, but is not limited to, one or more of recombinant mammalian Activin A, Alantolactone, or a combination thereof. In some embodiments, compositions disclosed herein can include medium having at least one Activin A inhibitor, where the at least one Activin A inhibitor can be one or more of A83-01, RepSox, D4476, Ly364947, R268712, SD208, SB505124, SM16, Galunisertib, SB525334, SB431542, or a combination thereof. In certain embodiments, compositions disclosed herein can include a medium having at least one BMP inhibitor, where the at least one BMP inhibitor can include, but is not limited to, one or more of LDN193189, LDN214117, IDN212854, DMH2, K02288, ML347, SGC AAK1-1, PD407824, UK383367, A01, recombinant mammalian Noggin and Dorsomorphin, non-mammalian Noggin and Dorsomorphin, or a combination thereof.

In certain embodiments and further to paragraphs [0004]-[0007] above, compositions disclosed herein can include, but are not limited to, at least one BMP signaling activator, at least one fibroblast growth factor receptor (FGFR) 3 activator, at least one Retinoic Acid signaling activator, and a protein enriched cell culture medium wherein the protein concentration can be about 0.5% (w/v) or more protein up to about 30% (w/v) protein (e.g., about 0.5% to about 20.0% (w/v)) and wherein the composition does not include a transforming growth factor-b (TGF-β) inhibitor. In other embodiments, compositions disclosed herein do not include transforming growth factor-b (TGF-β) inhibitor or transforming growth factor-b (TGF-β) activator. In accordance with these embodiments, compositions disclosed herein having a protein enriched culture medium (e.g., MENM alpha media) can further include at least one population of cells, including at least a population of definitive endodermal (DE) cells; a population of anterior foregut endoderm (AFE) cells, a population of ventral pharyngeal endodeim (VPE) cells, a population of pharyngeal endoderm (PE) cells, and a population of third pharyngeal pouch endoderm (TPPE) cells. In accordance with these embodiments, the at least one population of cells further includes a protein enriched media and one or more agents disclosed herein. In some embodiments, compositions disclosed herein can further include at least one Wnt activator.

In certain embodiments and further to paragraphs [0004]-[0008] above, compositions disclosed herein can include a media, a protein enriched media and can further include, but are not limited to, at least one of BMP signaling activator, at least one fibroblast growth factor receptor (FGFR) 3 activator, at least one fibroblast growth factor receptor (FGFR) 2 activator, at least one NOTCH signaling activator, and a protein enriched cell culture medium wherein the protein concentration can contain about 0.5% (w/v) or more protein up to about 30% (w/v) protein (e.g., about 0.5% to about 20.0% (w/v)) protein. In some embodiments, compositions disclosed herein can further include at least one Retinoic acid signaling activator, and/or at least one sonic hedgehog signaling activator. In accordance with these embodiments, compositions disclosed herein having a protein enriched culture medium (e.g., MEM alpha media) can further include at least one population of cells, including at least a population of definitive endodermal (DE) cells; a population of anterior foregut endoderm (AFE) cells, a population of ventral pharyngeal endodern (VPE) cells, a population of pharyngeal endoderm (PE) cells, and a population of third pharyngeal pouch endoderm (TPPE) cells. In some embodiments and further to the disclosures herein, compositions disclosed herein can further include at least one Wnt activator.

In certain embodiments and further to paragraphs [0004]-[0009] above, compositions disclosed herein can further include, but are not limited to, at least one of BMP signaling activator, at least one Retinoic acid receptor signaling activator, at least one inhibitor of TGF-β/Activin A signaling activator, at least one activator of sonic hedgehog signaling, and a protein enriched cell culture medium where the protein concentration can be about 0.5% (w/v) or more protein up to about 30% (w/v) protein (e.g., about 0.5% to about 20.0% (w/v)) protein. In accordance with these embodiments, compositions disclosed herein having a protein enriched culture medium (e.g., MEM alpha media) can further include at least one population of cells, including at least a population of definitive endodermal (DE) cells; a population of anterior foregut endoderm (AFE) cells, a population of ventral pharyngeal endoderm (VPE) cells, a population of pharyngeal endoderm (PE) cells, and a population of third pharyngeal pouch endoderm (TPPE) cells. In some embodiments, compositions disclosed herein can further include at least one Vascular Endothelial Growth Factor (VEGF) receptor signaling activator.

In some embodiments, and further to paragraphs [0004]-[0010] above, compositions disclosed herein having at least one BMP signaling activator can include having at least one of BMP4, or SB4. In some embodiments, compositions disclosed herein can include at least one FGFR3 activator. In accordance with these embodiment, at the least one FGFR3 activator can include at least one of FGF8, FGF1, FGF2, FGF9, Heparin, or a combination thereof. In some embodiments, compositions can further include at least one Retinoic Acid signaling activator. In accordance with these embodiments, compositions having at least one Retinoic Acid signaling activator can include one or more of Retinoic acid, Vitamin A, TTNPB, AC261066, SR1078, SR221, BMS493, Fenretinide, AM580, Adapalene, Ch55, or a combination thereof. In some embodiments, compositions disclosed herein can include at least one VEGF receptor signaling activator. In accordance with these embodiments, compositions disclosed herein having at least one VEGF receptor signaling activator can include at least one of VEGF, VEGF 165, multimeric VEGF, VEGF and a crosslinker, an anti-VEGF receptor antibody and an antibody crosslinker, or a combination thereof.

In certain embodiments and further to paragraphs [0004]-[0011] above, compositions disclosed herein can include a composition or a culture media having at least one FGF receptor 1 and at least one FGF receptor 2 signaling activator; at least one Retinoic Acid signaling activator; at least one inhibitor of BMP signaling; at least one inhibitor of sonic hedgehog signaling; at least one activator of Activin A signaling; in a protein enriched media where the protein concentration can be about 0.5% (w/v) or more protein up to about 30% (w/v) protein (e.g., about 0.5% to about 20.0% (w/v)) protein and where the composition does not include an activator of BMP signaling. In other embodiments, these compositions in a protein enriched media can further include at least one population of cells, the at least one population of cells can include definitive endoderm (DE) cells, anterior foregut endoderm (AFE) cells, pharyngeal endoderm (PE) cells, ventral pharyngeal endoderm (VPE) cells, third pharyngeal pouch endoderm (TPPE) cells, and/or thymic cells (e.g., thymic epithelial cells (TEC)). In some embodiments, compositions disclosed herein can further include at least one of Wnt-signaling activator, at least one NOTCH pathway signaling activator, at least one CD40 pathway signaling activator, at least one RANK signaling pathway activator, and/or at least one Ghrelin receptor signaling activator.

In some embodiments and further to paragraphs [0004]-[0012] above, compositions disclosed herein can further have at least one Wnt activator. In accordance with these embodiments, the at least one Wnt activator can include, but is not limited to, at least one of Wnt3a, Wnt4, CHIR 98014, AMBMP hydrochloride, or a combination thereof. In certain embodiments, compositions disclosed herein can include or further include at least one NOTCH pathway activator. In accordance with these embodiments, compositions disclosed herein having at least one NOTCH pathway activator can include, but are not limited to, Yhhu 3792, DLL1, DLL3, DLL4, Jagged 1, and Jagged 2. In certain embodiments, compositions disclosed herein can include or further include at least one CD40 pathway activator. In accordance with these embodiments, the at least one CD40 pathway activator can include, but are not limited to, CD40 ligand, multimeric CD40 ligand, CD40 ligand and a crosslinker, and an anti-CD40 antibody with an antibody crosslinker. In some embodiments, compositions disclosed herein can include or further include at least one RANK pathway activator. In accordance with these embodiments, the at least one RANK pathway activator can include. but are not limited to, soluble RANK ligand, RANK ligand, multimeric RANK ligand, RANK ligand and a crosslinker, and an anti-RANK antibody with an antibody crosslinker. In other embodiments, compositions disclosed herein can further include at least one Ghrelin receptor pathway activator. In accordance with the embodiments, the at least one Ghrelin receptor pathway activator can include, but are not limited to, Ghrelin, MK 0677, Tabimorelin hemifumerate, and L-692,585. In certain embodiments, compositions disclosed herein can include at least one NOTCH pathway activator and at least one of at least one CD40 pathway activator and at least one RANK pathway activator. In accordance with these embodiments, compositions disclosed herein having a protein enriched culture medium (e.g., MEM alpha media) and one or more agents identified herein. In other embodiments, these compositions can further include at least one population of cells, including at least a population of definitive endodermal (DE) cells; a population of anterior foregut endoderm (AFE) cells, a population of ventral pharyngeal endoderm (VPE) cells, a population of pharyngeal endoderm (PE) cells, and a population of third pharyngeal pouch endoderm (TPPE) cells. In accordance with these embodiments, the at least one population of cells further includes a protein enriched media and two or more, three or more, four or more agents disclosed herein.

In some embodiments and further to paragraphs [0004]-[0013] above, compositions disclosed herein having at least one FGF receptor 1 and FGF receptor 2 signaling activator can include, but is not limited to, at least one of FGF10, FGF3, FGF4, FGF7, FGF22, or a combination thereof. In some embodiments, compositions disclosed herein including at least one Retinoic Acid signaling activator can include but is not limited to, at least one of Retinoic acid, Vitamin A, TTNPB, AC261066, SR1078, SR221, BMS493, Fenretinide, AM580, Adapalene, Ch55, or a combination thereof. In some embodiments, compositions disclosed herein having at least one BMP inhibitor can include, but are not limited to at least one of LDN193189, LDN214117, LDN212854, DMH2, K02288, ML347, SGC AAK1 1, PD407824, UK383367, A01, recombinant mammalian Noggin and Dorsomorphin, non-mammalian Noggin and Dorsomorphin, or a combination thereof. In some embodiments, compositions disclosed herein having at least one inhibitor of sonic hedgehog signaling can include, but is not limited to, at least one of SANT1, SANT2, Ul 866A, Dynapyrazole-A, Dynarrestin, Cyclopamine, HIP1, GANT58, AY9944 dihydrochloride, RU-SKI 43 hydrochloride, or a combination thereof. In some embodiments, compositions disclosed herein including at least one Activin A activator can include, but is not limited to, at least one of recombinant mammalian Activin A, SB4, Alantolactone, or a combination thereof. In some embodiments, compositions disclosed herein including at least one NOTCH signaling activator can include, but is not limited to, at least one of Yhhu 3792, DLL1, DLL3, DL L4, Jagged 1, and Jagged 2 or a combination thereof. In some embodiments, compositions disclosed herein having at least one Wnt signaling activator can include, but are not limited to Wnt3a, Wnt4, CHIR 98014, and AMBMP hydrochloride or a combination thereof.

In certain embodiments and further to paragraphs [0004]-[0014] above, activating agents supplemented in base medias (e.g., absent of protein), with low to medium levels of protein or in protein enriched medias disclosed herein can have a concentration of about 1 nM to 10 LM depending on what is being activated, the agent(s) of interest and the agent inducing activation. One of skill in the relevant art will understand these distinctions to activate the desired target or system disclosed herein (e.g., NOTCH, Wnt, RA or molecule or another pathway). In certain embodiments, activating agents supplemented in medias absent of protein in a base media disclosed herein can have a concentration of about 1 nM to 10 μM depending on what is being activated, the agent(s) of interest and the agent inducing activation. In some embodiments, based media disclosed herein can further include a protein supplement in the form of a protein supplement or for example, serum (e.g., Fetal Bovine Serum (FBS), Bovine Serum Albumin (BSA), human serum, knock out serum of the like).

In certain embodiments and further to paragraphs [0004]-[0015] above, methods for differentiating mammalian pluripotent stem cells (PSCs) are disclosed. In some embodiments, the present disclosure provides methods for differentiating mammalian PSCs into anterior primitive streak cells and subsequently definitive endoderm (DE) cells. In certain embodiments, methods for differentiating mammalian PSCs into anterior primitive streak cells and subsequently DE cells can include incubating mammalian PSCs or anterior primitive streak cells or DE c ells with compositions disclosed herein, generating DE cells that can have capacity to differentiate into at least one of AFE, VPE, TPPE, PE, and/or thymic cells.

In some embodiments and further to paragraphs [0004]-[0016] above, early incubation times can include cell culture compositions having protein concentrations of less than 0.5% and after a predetermined period switched to cell culture compositions having at least 0.5% (w/v) or more protein up to about 30.0% (w/v) (e.g., about 0.5% to about 20.0% (w/v)) protein concentration for enriched protein conditions. In some embodiments, methods can include incubating mammalian PSCs or anterior primitive streak cells or DE cells for about 12 hours up to about 6 days in a low-protein or protein-free medium, wherein the low-protein medium can have a concentration of less than 0.5% (w/v) protein in the medium. In certain embodiments, methods disclosed herein can further include incubating PSCs or APS cells in a medium having a concentration of protein of less than 0.5% (w/v) protein for about 12 hours up to about 4 days, where the medium can further include, but not be limited to, at least one of a BMP inhibitor and at least one of an Activin inhibitor, to generate DE cells with the capacity to efficiently differentiate into AFE cells, VPE cells, TPPE cells, PE cells, and/or thymic cells. In other embodiments, culturing compositions for methods disclosed herein can include at least one of a BMP inhibitor and at least one of an Activin inhibitor, to generate DE cells from PSCs or APS cells with the capacity to efficiently differentiate into AFE cells, VPE cells, TPPE cells, PE cells, and/or thymic cells (TEP or TEC cells). In other embodiments, cell culturing compositions of use in methods disclosed herein can include a media and at least one of an Activin activator, to generate DE cells from PSCs or APS cells with the capacity to efficiently differentiate into AFE cells, VPE cells, TPPE cells, PE cells, and/or thymic cells (TEP or TEC cells).

In certain embodiments and further to paragraphs [0004]-[0017] above, methods and compositions disclosed herein can include incubating mammalian PSCs, or APS cells, or DE cells, or AFE cells, or VPE cells, or PE cells, or TPPE cells, or thymic cells in any of the compositions disclosed herein and supplementing the composition with ITS (insulin, transferrin, and selenium), ITSX (insulin, transferrin, and selenium and ethanolamine), ITSE or the like from about day 0 to up to 24 hours of incubation at a dilution of about 1:50 to about 1:5000. In some embodiments, methods disclosed herein can include supplementing the composition with ITS or ITS-X, where the ITS or the ITS-X can be about 1:2000 or increased to about 1:2000. In certain embodiments, methods disclosed herein can further include incubating mammalian PSCs or anterior primitive streak cells or DE cells in a medium that can include one or more of epidermal growth factor (EGF), hydrocortisone, Vitamin C, Vitamin E, non-essential amino acids, sodium pyruvate, glutamine, trace elements, lipids and/or beta mercaptoethanol or any combination thereof.

In certain embodiments and further to paragraphs [0004]-[0018] above, methods disclosed herein can further include incubating DE or AFE cells in a replacement composition or replacement media having a concentration of protein of at least about 0.5% (w/v) or more protein up to about 30.0% (w/v) protein (e.g., about 0.5% to about 20.0% (w/v)) protein for about 12 hours up to about 4 days, where the replacement composition can include at least one of: at least one FGF receptor 3 signaling activator, at least one Retinoic Acid signaling activator, and at least one activator of BMP signaling activator for about 12 hours up to about 14 days to produce at least one of AFE cells and/or VPE cells with the capacity to efficiently differentiate into TEP and/or TEC cells. In accordance with these embodiments, methods disclosed herein can further include replacing the medium with a medium having a concentration of protein can be about 0.5% (w/v) or more protein up to about 30.0% (w/v) protein (e.g., about 0.5% to about 20.0% (w/v)) protein, where the medium can further include at least one of: at least one FGF receptor 1 and FGF receptor 2 signaling activator, at least one Retinoic Acid signaling activator, at least one inhibitor of BMP signaling, at least on inhibitor of sonic hedgehog signaling, at least one Activin A activator and at least one Wnt pathway signaling activator for about 12 hours up to about 60 days or more. In accordance with these embodiments, methods disclosed herein can yield a final population of cells that include thymic cells, wherein the thymic cells can be at least one of TEP and TEC cells.

In certain embodiments and further to paragraphs [0004]-[0019] above, methods disclosed herein can further include replacing the composition with a replacement composition having about 0.5% (w/v) or more protein up to about 30.0% (w/v) protein (e.g., about 0.5% to about 20.0% (w/v)) and the replacement composition containing at least one of: at least one FGF receptor 1 and FGF receptor 2 signaling activator, at least one Retinoic Acid signaling activator, at least one inhibitor of BMP signaling, at least one inhibitor of sonic hedgehog signaling and at least one Activin A activator and incubating the DE, AFE and/or VPE cells for about 12 hours up to about 60 days or more to generate thymic cells (e.g. TEC and/or TEP cells).

In some embodiments and further to paragraphs [0004]-[0020] above, methods and compositions disclosed herein can include differentiating at least about 70% up to about 100% of mammalian PSCs, anterior primitive streak cells, and/or DE cells into AFE cells that are SOX2+, FOXA2+ and SOX 17− able to further differentiate into thymic cells. In some embodiments, methods disclosed herein include differentiating at least about 60% up to about 100% of the mammalian AFE cells into VPE cells that are HOXA3+HOXB1−NKX2.1−. In some embodiments, compositions and methods disclosed herein can include at least about 5% or more, 10% or more, 15% or more, or 20%. Or 30%, or 40% or more of mammalian AFE or VPE cells differentiated into thymic cells (e.g., TEPs and TECs). In accordance with these embodiments, thymic cells created or differentiated using compositions and methods disclosed herein include cells that are EPCAM+, CD104+, CD205+ and MHC-II+.

In certain embodiments and further to paragraphs [0004]-[0021] above, the present disclosure provides kits containing one or more composition disclosed herein and at least one container. In some embodiments, kits can include mammalian PSCs or components needed to harvest mammalian PSCs. In some embodiments, kits disclosed herein can include one or more agents for supplementing a composition or medium disclosed herein and optionally, the medium or composition or replacement composition disclosed herein. In certain embodiments, the present disclosure provides kits for differentiating cells into functioning thymic cells disclosed herein for use to treat or prevent a health condition. In certain embodiments, kits can include kits for storage, transport, and use in ameliorating, preventing and/or treating a health condition in a subject.

In certain embodiments and further to paragraphs [0004]-[0022] above, the present disclosure provides pharmaceutical compositions and methods of treating a health condition in a subject using a pharmaceutically acceptable composition disclosed. In certain embodiments, the pharmaceutical composition includes one or more cell populations generated by compositions and methods of the instant disclosure. In some embodiments, methods disclosed herein include treating an immune-related health condition in a subject by administering a cell population generated by any one of the compositions and methods disclosed herein to a subject in need thereof. In some embodiments, methods disclosed herein can treat a health condition in a subject by administering thymic cells generated by any one of the methods disclosed herein to a subject in need thereof.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIG. 1 illustrates an exemplary schematic of methods and compositions disclosed herein for generating intermediary cells and thymic cells from pluripotent stem cells in accordance with certain embodiments of the present disclosure.

FIG. 2 illustrates a decoding chart of agents, modulators, additives, and base mediums used in compositions and methods disclosed herein in accordance with certain embodiments of the present disclosure.

FIGS. 3A-3C illustrate compositions and methods for AFE and VPE induction (3A) from various cells and conditions and representative flow plots and comparative quantification of SOX17⁻SOX2⁺ cell production (3B) under various growth conditions and flow plots and FOXA2⁺SOX2⁺ cell production (3C) under various growth conditions in accordance with certain embodiments of the present disclosure.

FIGS. 4A-4E illustrate compositions and methods for AFE, VPE and thymic epithelial progenitor (TEP) cell induction (4A) from various cells and conditions and representative comparative quantification of CD205⁺EPCAM⁺ cells (4B) under conditions to a selected day and analysis of for HOXA3 (4C), KRT8 (4D), and FOXN1 (4E) comparative gene expression under various differentiation protocols in accordance with certain embodiments of the present disclosure.

FIGS. 5A-5C illustrate compositions and methods for AFE and VPE production (5A) and a representative flow plot and comparative quantification of SOX17⁻SOX2⁺ cells (5B) under various conditions at a selected time and a representative flow plot and comparative quantification of FOXA2⁺SOX2⁺ cells (5C) under various conditions at a selected time from different differentiation protocols in accordance with certain embodiments of the present disclosure.

FIGS. 6A-6G illustrate compositions and methods for AFE, VPE and thymic epithelial progenitor (TEP) cell induction (6A) and a representative flow plot (6B) and quantification of EPCAM+/CD104^{hi} cells (6C) and comparative analysis of gene expression of FOXN1 (6D), KRT5 (6E), NKX2 (6F), and NKX2-1 (6G) gene expression from each differentiation protocol in accordance with certain embodiments of the present disclosure.

FIG. 7 illustrates an exemplary experimental set-up in accordance with certain embodiments of the present disclosure,

FIGS. 8A-8B illustrate additional stimulants used to enhance thymic cell production having improved marker representation, 8A and 8A illustrate percent representative marker outcome under the varying conditions in accordance with certain embodiments of the present disclosure.

FIGS. 9A-9B illustrate exemplary results from qPCR detection of representative markers under control and experimental conditions in accordance with certain embodiments of the present disclosure.

FIG. 10 is a schematic representation of a derivation of a stem cell-derived thymic organoid (sTO) in accordance with certain embodiments of the present disclosure.

FIGS. 11A-11C illustrate exemplary results under various conditions of experimental thymic cell derivation protocols. 11A illustrates exemplary flow cytometry results; 11B illustrates production of sTOs compared to production of various T cell populations, and 11C represents TEP versus sTO under certain experimental conditions accordance with certain embodiments of the present disclosure.

### DEFINITIONS

Terms, unless defined herein, have meanings as commonly understood by a person of ordinary skill in the art relevant to certain embodiments disclosed herein or as applicable.

As used herein “about” unless otherwise indicated, applies to all numbers expressing quantities of agents and/or compounds, properties such as molecular weights, reaction conditions, and as disclosed herein are contemplated as being modified in all instances by this term. Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals.

As used herein, “treat,” “treating,” or “treatment” can mean reversing, ameliorating, or inhibiting onset or inhibiting progression of a health condition or disease or a symptom of the health condition or disease. In other embodiments, a health condition can be prevented or the risk of onset be ameliorated.

As used herein, “pluripotent stem cell” or “pluripotent cell” can refer to a cell capable, under appropriate conditions, of producing progeny of several different cell types that are derivatives of all of the three germinal layers (i.e., endoderm, mesoderm, and ectoderm) or reprogrammed. Examples of pluripotent stem cells (PSC) include, but are not limited to, embryonic stem (ES) cells, embryonic germ stem (EG) cells, induced pluripotent stem (iPSC) cells, adult stem cells, and the like. PSC cells can be from any organism of interest, including but not limited to, primate, (e.g., human), canine, feline, murine, equine, porcine, avian, camel, bovine, ovine, and the like.

As used herein, “marker” can refer to any molecule that can be measured or detected, for example. In certain embodiments disclosed herein, a marker can include, without limitations, a nucleic acid, such as, a transcript of a gene, a polypeptide product of a gene, a polypeptide, a protein, a glycoprotein, a carbohydrate, a glycolipid, a lipid, a lipoprotein, a carbohydrate, and/or a small molecule. As used herein, “expression” and grammatical equivalents thereof, in the context of a marker, can refer to production or transcription or translation of the marker. In addition, level or amount of the marker can be assessed and compared to controls in order to evaluate a process.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the present disclosure relate to novel compositions and methods for generating thymic cells from pluripotent stem cells and intermediary cells thereof. A critical issue for accelerating research and treatment efforts related to thymus function and T-cell development has been the lack of an effective and efficient differentiation protocol for pluripotent stem cell lines. Current differentiation protocols produce a small number of mature thymic cells with a high percentage of immature T cells having aberrant phenotypes that are not useful for study or for therapeutic use. In addition, negative selection, the process of removing autoreactive T cells during thymic T cell education, is currently not feasible. Therefore, there is a need in the art for improved compositions, conditions, and methods for generating thymic cells from pluripotent stem cells. In certain embodiments, novel and efficient compositions and methods for generating thymic cells (e.g., TEC and TEP cells) and intermediary cells (e.g., DE, AFE, VPE cells) from pluripotent stem cells (PSCs) are disclosed herein where functional thymic cells can generate conventional T cells suitable for clinical applications, including, but not limited to, adoptive cell therapy, immune therapies, and cancer therapies, for example.

Appendix A filed with the priority application is incorporated herein by reference in its entirety for all purposes. One of skill in the art understands that there are several causes of decline of the thymus and naïve T cells. For example, thymic atrophy (e.g., involution, immunosenescence), chemotherapeutic side effects, graft versus host disease (GvHD) and human immunodeficiency virus (HIV) can cause these declines. State of the art thymic epithelial cell differentiation protocols are deficient in functional TECs after long periods of time in vivo, low numbers of FOXN1 positive cells in vitro, low expression levels of TEP/TEC markers in vivo and in vitro providing a need for improved methods of generating these cells for therapeutic use and restoration. Compositions and methods disclosed herein provide for improved methods of producing functional thymic cells using novel supplemented medias, for example. In certain embodiments, early thymic progenitor signaling can enhance expression of FOXN1 during differentiation of thymic epithelial cells (TECs), for example CD40 ligands, RANK ligands and NOTCH Ligands. In some embodiments disclosed herein, it is demonstrated that ETP-derived signals (e.g., CD40L, RANKL, and NOTCH) can enhance iPSC-derived TEP expression of FOXN1 and MHC-II. In other embodiments, compositions and methods disclosed herein can be used to form stem cell-derived thymic organoids, produce some single positive T cell and provide for further TEP/TEC maturation. In other embodiments, optimized TEP/TEC protocols disclosed herein can be combined with the stem cell-derived thymic organoid systems for improved outcome such as thymus function restoration.

In certain embodiments and further to paragraph [0043]-[0044] above, compositions disclosed herein can be used to provide culturing conditions for generating thymic cells (e.g., TEC and TEP cells) and intermediary cells (e.g., DE, AFE, VPE cells) from PSCs according to methods of the present disclosure. In some embodiments, compositions disclosed herein can include a culturing media. As used herein, the term “medium” or “media” is used in context of cell culture or the phrase “cell culture medium” or “cell medium,” “replacement composition” or composition as appropriate can refer to a cellular growth medium suitable for culturing of cells of the present disclosure. A “base medium,” as referred to herein of use as a low protein or enriched protein-containing medium, can be a cell culture medium that has not been modified by adding one or more additives/supplements. Non-limiting examples of a base medium suitable for use herein can include MEM alpha media, Minimum Essential Medium (MEM), Eagle's Medium, Dulbecco's Modified Eagle Medium (DMEM), Dulbecco's Modified Eagle Medium: Nutrient Mixture F-12 (DMEM/F12), F10 Nutrient Mixture, Ham's F10 Nutrient Mix, Ham's F12 Nutrient Mixture, Medium 199, RPMI, RPMI 1640, reduced serum medium, basal medium (BME), DMEM/F12 (1:1), Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E and the like, and combinations thereof. In some embodiments, compositions disclosed herein can include a base medium that contains one or more additives as disclosed herein.

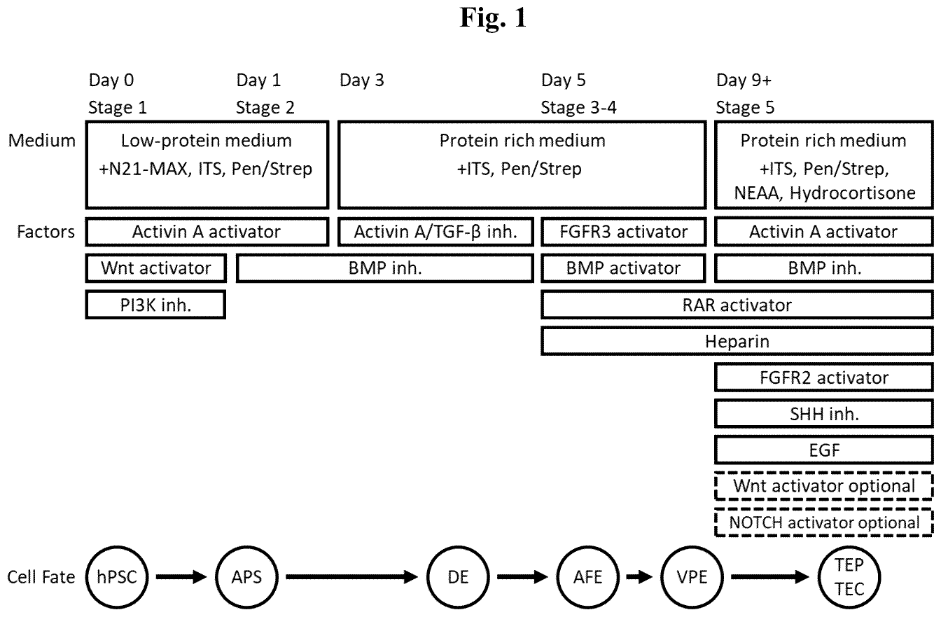

In certain embodiments and further to paragraphs [0043]-[0045] above, compositions disclosed herein can be used in one or more stages of differentiation for producing thymic cells from PSCs. As illustrated in one exemplary schematic depicted in FIG. 1, producing thymic cells from PSCs according to the present disclosure can include several stages of differentiation. In this example, five exemplary stages of differentiation are illustrated in order to appreciate compositions and methods disclosed herein where: (1) culturing PSCs to generate anterior primitive streak (APS) cells; (2) culturing APS cells to generate definitive endoderm (DE) cells; (3) culturing DE cells to generate anterior foregut endoderm (AFE) cells; (4) culturing AFE cells to generate ventral pharyngeal endodermal (VPE) cells; and (5) culturing VPE cells to generate thymic cells (e.g., thymic epithelial progenitor (TEP) cells, thymic epithelial cells (TECs), or a combination thereof) are described herein.

### Stage 1—Culturing PSCs to Generate APS Cells

In certain embodiments, the present disclosure provides compositions for use in vitro differentiation of pluripotent stem cells (PSCs) into thymic cells and intermediary cells thereof. In some embodiments, PSCs used in present disclosure can include, but are not limited to, embryonic stem cells, embryonic germ cells, or induced pluripotent stem cells (iPSCs). In some embodiments, PSCs can be mammalian PSCs. In other embodiments, PSCs can be human PSCs. In other embodiments, PSCs used in present disclosure can be isolated from an autologous source. As used herein, the term “autologous” refers to obtaining PSCs from the same subject to be treated and/or monitored with the thymic cells generated as disclosed herein. In some embodiments, PSCs of use in embodiments of the present disclosure can be isolated from an allogeneic source. As used herein, the term “allogeneic” refers to obtaining PSCs from a different subject than the subject to be treated and/or monitored with thymic cells generated as disclosed herein. In some embodiments, PSCs used in present disclosure can be harvested, generated, cultured, and/or characterized using standard methods in the art.

In certain embodiments, compositions disclosed herein can be used to provide conditions for culturing PSCs to generate APS cells according to methods of the present disclosure. In accordance with these embodiments, compositions disclosed herein can include a base medium having a low concentration of protein. In certain embodiments, a protein used in a base medium as disclosed herein can be serum or other protein supplement. In some embodiments, a protein used in a base medium as disclosed herein can be serum from a mammalian source (e.g., bovine, human) or protein derived from any source.

In some embodiments, compositions disclosed herein of use for culturing PSCs to generate APS cells according to methods of the present disclosure can be a base medium with low protein concentrations of about 0.5% (w/v) or less. In some embodiments, a base medium with low protein concentrations for use herein can have protein concentrations of less than about 0.01% (w/v), less than about 0.05% (w/v), less than about 0.1% (w/v), less than about 0.2% (w/v), less than about 0.3% (w/v), less than about 0.4% (w/v) or less than about 0.5% (w/v).

In some embodiments, compositions disclosed herein for culturing PSCs to generate APS cells according to methods of the present disclosure can include a base medium with at least one additive capable of modulating Wnt and/or a Wnt signaling pathway. As used herein, the term “modulating” means activating, propagating, inhibiting, upregulating the expression of, downregulating the expression of, and/or otherwise modifying the activity of a signaling pathway or a component of a signaling pathway. In accordance with these embodiments, compositions disclosed herein for culturing PSCs to generate APS cells according to methods of the present disclosure can be a base medium with at least one Wnt modulating agent. As used herein, “a Wnt modulating agent” can refer to any chemical (e.g., small molecule or other chemical agent), compound, polypeptide, protein, or fragment thereof, polynucleotide (DNA or RNA), or other agent that modulates Wnt and/or a Wnt signaling pathway. In some embodiments, compositions disclosed herein for culturing PSCs to generate anterior primitive streak (APS) cells according to methods of the present disclosure can be a base medium with at least one Wnt modulating agent capable of activating Wnt and/or a Wnt signaling pathway (e.g., “a Wnt activator”). In certain embodiments, an agent capable of modulating Wnt can be an activating agent. Non-limiting examples of Wnt activators suitable for uses disclosed herein can include DNA encoding β-catenin (e.g., naked DNA encoding β-catenin, plasmid expression vectors encoding β-catenin, viral expression vectors encoding β-catenin), β-catenin polypeptides, one or more Wnt/β-catenin pathway agonists (e.g., Wnt ligands, DSH/DVL-1, -2, -3, LRP6N, WNT3A, WNT5A, and WNT3A, 5A), one or more glycogen synthase kinase 3 β (GSK3 (3) inhibitors (e.g., lithium chloride (LiCl), Purvalanol A, olomoucine, alsterpaullone, kenpaullone, benzyl-2-methyl-1,2,4-thiadiazolidine-3,5-dione (TDZD-8), 2-thio(3-iodobenzyl)-5-(1-pyridyl)-[1,3,4]-oxadiazole (GSK3 inhibitor II), 2,4-dibenzyl-5-oxothiadiazolidine-3-thione (OTDZT), (2′Z,3′E)-6-Bromoindirubin-3′-oxime (BIO), α-4-Dibromoacetophenone (i.e., Tau Protein Kinase I (TPK I) Inhibitor), 2-Chloro-1-(4,5-dibromo-thiophen-2-yl)-ethanone, N-(4-Methoxybenzyl)-N′-(5-nitro-1,3-thiazol-2-yl)urea (AR-A014418), indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5,5′-dibromoindirubin; 5-nitroindirubin; 5-chloroindirubin; 5-methylindirubin, 5-bromoindirubin, 4-Benzyl-2-methyl-1,2,4-thiadiazolidine-3,5-dione (TDZD-8), 2-thio(3-iodobenzyl)-5-(1-pyridyl)-[1,3,4]-oxadiazole (GSK3 inhibitor II), 2,4-Dibenzyl-5-oxothiadiazolidine-3-thione (OTDZT), (2′Z,3′E)-6-Bromoindirubin-3′-oxime (BIO), a-4-Dibromoacetophenone (i.e., Tau Protein Kinase I (TPK I) Inhibitor), 2-Chloro-1-(4,5-dibromo-thiophen-2-yl)-ethanone, (vi) N-(4-Methoxybenzyl)-N′-(5-nitro-1,3-thiazol-2-Aurea (AR-A014418), H-KEAPPAPPQSpP-NH2 (L803) and Myr-N-GKEAPPAPPQSpPNH2 (L803-mts)), one or more anti-sense RNA or siRNA that bind specifically to GSK3β mRNA, one or more casein kinase 1 (CK1) inhibitors (e.g., antisense RNA or siRNA that binds specifically to CK1 mRNA), and the like.

In some embodiments, compositions disclosed herein for culturing PSCs to generate APS cells according to methods of the present disclosure can be a base medium with at least one Wnt activator at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, for example. In some embodiments, compositions disclosed herein for culturing PSCs to generate APS cells according to methods of the present disclosure can be a base medium with at least one Wnt activator at a concentration of about 5 ng/ml to about 200 ng/ml (e.g., about 10 ng/ml to about 150 ng/ml, about 15 ng/ml to about 125 ng/ml or about 15 ng/ml to about 100 ng/ml or about 15 ng/ml to about 50 ng/ml). In some embodiments, compositions disclosed herein for culturing PSCs to generate APS cells according to methods of the present disclosure can be made up of a base medium with at least one Wnt activator at a concentration of about 5 ng/ml, about 10 ng/ml, about 25 ng/ml, about 50 ng/ml, about 75 ng/ml, about 100 ng/ml, about 125 ng/ml, about 150 ng/ml, about 175 ng/ml, or about 200 ng/ml.

In some embodiments, compositions disclosed herein for culturing PSCs to generate APS cells according to methods of the present disclosure can be a base medium with at least one Activin A modulating agent (and optionally, at least one Wnt activator and/or at least one BMP inhibitor). As used herein, “an Activin A modulating agent” can refer to any chemical (e.g., small molecule or other chemical agent), compound, polypeptide, protein, or fragment thereof, polynucleotide (DNA or RNA), or other agent that modulates Activin A and/or an Activin A signaling pathway. In some embodiments, compositions disclosed herein for culturing PSCs to generate APS cells according to methods disclosed herein can include a base medium with at least one Activin A activator. In accordance with these embodiments, an Activin A activator can be Activin A. and/or variants thereof or functional analogs thereof. Non-limiting examples of Activin A analogs suitable for use disclosed herein can include IDE1 (2-[6-carboxy-hexanoyl)-hydrazonomethyl]-benzoic acid), IDE2 (7-(2-cyclopentylidenehydrazino)-7-oxoheptanoic acid, and the like. In some embodiments, compositions disclosed herein can have at least one Activin A activator that can include at least one of recombinant mammalian Activin A, SB4, Alantolactone, or a combination thereof.

In some embodiments, compositions disclosed herein for culturing PSCs to generate APS cells according to methods of the present disclosure can include a base medium with at least one Activin A activator (and optionally, at least one Wnt activator and/or at least one BMP inhibitor) at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, for example. In some embodiments, compositions disclosed herein for culturing PSCs to generate APS cells according to methods of the present disclosure can be a base medium with at least one Activin A activator (and optionally, at least one Wnt activator and/or at least one BMP inhibitor) at a concentration of about 5 ng/ml to about 200 ng/ml (e.g., about 10 ng/ml to about 75 ng/ml or about 15 ng/ml to about 50 ng/ml). In some embodiments, compositions disclosed herein for culturing PSCs to generate APS cells according to methods of the present disclosure can include a base medium with at least one Activin A activator (and optionally, at least one Wnt activator and/or at least one BMP inhibitor) at a concentration of about 5 ng/ml, about 10 ng/ml, about 25 ng/ml, about 50 ng/ml, about 75 ng/ml, about 100 ng/ml, about 125 ng/ml, about 150 ng/ml, about 175 ng/ml, or about 200 ng/ml. In certain embodiments, the base media is a low protein-containing (e.g., 1% (w/v) protein or less) media.

In some embodiments, compositions disclosed herein for culturing PSCs to generate APS cells according to methods of the present disclosure can be a base medium with at least one phosphoinositide 3-kinase (PI3K) modulating agent (and optionally, at least one Activin A inhibitor, at least one Wnt activator and/or at least one BMP inhibitor). As used herein, “a PI3K modulating agent” can refer to any chemical (e.g., small molecule or other chemical agent), compound, polypeptide, protein or fragment thereof, polynucleotide (DNA or RNA), or other agent that modulates PI3K and/or a PI3K signaling pathway. In some embodiments, compositions disclosed herein for culturing PSCs to generate APS cells according to methods of the present disclosure can be a base medium with at least one PI3K inhibitor. Non-limiting examples of PI3K inhibitors suitable for use in compositions and methods disclosed herein can include BKM120, BEX235, BGT226, Idelalisib, GDC-0941, IPI-145 (INK1197), GSK2636771, PI-103, BEZ235, BGT226, VS-5584m, (SB2343), PI-103, ZSTK474, GSK1059615, Gedatolisib, HS-173, Alpelisib (BYL719), PIK-75, A66, YM201636, TGX-221, GSK2636771, CZC24832, AS-252424, AS-604850, CAY10505, CAL-101 (Idelalisib, GS-1101), PIK-294, PI-3065, PIK-293, IC-87114, AS-605240, PIK-90, other P3K inhibitor and the like and combinations thereof.

In some embodiments, compositions disclosed herein for culturing PSCs to generate APS cells according to methods of the present disclosure can be a base medium with at least one PI3K inhibitor (and optionally, at least one Activin A inhibitor, at least one Wnt activator and/or at least one BMP inhibitor) at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, for example. In some embodiments, compositions disclosed herein for culturing PSCs to generate APS cells according to methods of the present disclosure can include a base medium with at least one PI3K inhibitor at a concentration of about 5 ng/ml to about 200 ng/ml (e.g., about 10 ng/ml to about 75 ng/ml or about 15 ng/ml to about 50 ng/ml for example). In some embodiments, compositions disclosed herein for culturing PSCs to generate APS cells according to methods disclosed herein can include a base medium with at least one PI3K inhibitor at a concentration of about 5 ng/ml, about 10 ng/ml, about 25 ng/ml, about 50 ng/ml, about 75 ng/ml, about 100 ng/ml, about 125 ng/ml, about 150 ng/ml, about 175 ng/ml, or about 200 ng/ml.

In certain embodiments, compositions disclosed herein for culturing PSCs to generate APS cells according to methods of the present disclosure can be a base medium with a low concentration of protein (e.g., 0.5% (w/v) protein or less) and at least one of: at least one Wnt modulating agent, at least one Activin A modulating agent, at least one PI3K modulating agent, or a combination thereof. In some embodiments, compositions disclosed herein for culturing PSCs to generate APS cells according to methods disclosed herein can include a base medium with a low concentration of protein and at least one of: at least one Wnt activator, at least one Activin A activator, at least one PI3K inhibitor, or a combination thereof. In some embodiments, compositions disclosed herein for culturing PSCs to generate APS cells according to methods of the present disclosure can be a base medium with a low concentration of protein, and at least one Wnt activator, at least one Activin A activator, and at least one PI3K inhibitor and; optionally other culturing agents.

In certain embodiments, compositions disclosed herein for culturing PSCs to generate APS cells can be provided to the cell culture and incubated for a period of time suitable for PSCs cells to differentiate into APS cells. In some embodiments, culture condition disclosed herein can be from about 12 hours to about 48 hours or to about 72 hours or more. In some embodiments, compositions disclosed herein for culturing PSCs to generate APS cells can be provided to the cell culture and incubated for a period suitable for at least about 70% to at least about 99% up to 100% of the PSCs cells to differentiate into APS cells. In some embodiments, compositions disclosed herein for culturing PSCs to generate APS cells can be provided to the cell culture and incubated for a period suitable for about 70%, about 75%, about 80%, about 85%, about 90%, about 95%, or about 99% of the PSCs cells to differentiate into APS cells. In some embodiments, compositions disclosed herein for culturing PSCs to generate APS cells can be provided to the cell culture and incubated for a period of about 12 hours to about 5 days. In some embodiments, compositions disclosed herein for culturing PSCs to generate APS cells can be provided to the cell culture and incubated for a period of 12 hours or less or less than about 1 day, about 1 day, about 2 days, about 3 days, about 4 days, or about 5 days or any time in between.

### Stage 2—Culturing APS Cells to Generate DE Cells

In certain embodiments, compositions disclosed herein can be used to provide conditions for culturing APS cells to generate DE cells according to methods of the present disclosure. In accordance with these embodiments, compositions disclosed herein can be a base medium with a low concentration of protein (e.g., a “low-protein medium”) or a high concentration of protein (e.g., a “protein enriched medium”). In some embodiments, compositions disclosed herein can be used to provide conditions for culturing APS cells to generate DE cells according to methods of the present disclosure can be a base medium with a high protein concentrations of about 0.5% (w/v) or more protein up to about 30% (w/v) protein (e.g., about 0.5% to about 20.0% (w/v)) protein. In some embodiments, a base medium with high protein concentrations of use as culturing media disclosed herein can have protein concentrations of more than about 0.5% (w/v), more than about 5.0% (w/v), more than about 10% (w/v), more than about 15% (w/v), more than about 20% (w/v), more than about 25% (w/v), up to about 30.0% (w/v). In some embodiments, a base medium with high protein concentrations of use as culturing media disclosed herein can have protein concentrations of at least about 0.5% (w/v) up to 30% (w/v) protein. In some embodiments, a base medium with high protein concentrations for use herein can have a protein concentration ranging from about 0.5% (w/v) to about 30.0% (w/v) (e.g., about 0.5% (w/v), about 5.0% (w/v), about 10% (w/v), about 15% (w/v), about 20% (w/v), about 25% (w/v), about 30% (w/v)).

In certain embodiments, compositions disclosed herein for culturing APS cells to generate DE cells according to methods of the present disclosure can include a base medium with at least one bone morphogenetic protein (BMP) modulating agent. As used herein, “a BMP modulating agent” can refer to any chemical (e.g., small molecule or other chemical agent), compound, polypeptide, protein, or fragment thereof, polynucleotide (DNA or RNA), or other agent that modulates BMP and/or a BMP signaling pathway. In some embodiments, compositions disclosed herein for culturing APS cells to generate DE cells according to methods of the present disclosure can be a base medium with at least one BMP inhibitor. Non-limiting examples of BMP inhibitors suitable for uses and supplements in enriched protein media disclosed herein can include, but is not limited to, LDN193189, LDN214117, LDN212854, DMH2, K02288, ML347, SGC AAK11, PD407824, UK383367, and A01, recombinant mammalian Noggin and Dorsomorphin, non-mammalian Noggin and Dorsomorphin, and the like. Any BMP inhibitor is contemplated of use herein to reduce or inhibit the BM P pathway. In some embodiments, compositions disclosed herein for culturing APS cells to generate DE cells according to methods of the present disclosure can be a base medium with at least one BMP inhibitor. In some embodiments, compositions disclosed herein for culturing APS cells to generate DE cells according to methods of the present disclosure can be a base medium with at least one BMP inhibitor at a concentration of at least about 5 ng/ml, at least about 10 ng/ml, at least about 15 ng/ml, at least about 25 ng/ml, at least about 50 ng/ml, at least about 75 ng/ml, at least about 100 ng/ml, at least about 200 ng/ml, at least about 250 ng/ml, at least about 300 ng/ml, at least about 400 ng/ml, at least about 500 ng/ml, or at least about 1000 ng/ml. In some embodiments, compositions disclosed herein for culturing APS cells to generate DE cells according to methods of the present disclosure can be a base medium having a high protein concentrations of at least 1.0% (w/v) or more and further include at least one BMP inhibitor at a concentration of about 5 ng/ml to about 350 ng/ml (e.g., about 10 ng/ml to about 150 ng/ml, about 15 ng/ml to about 100 ng/ml, or about 15 ng/ml to about 50 ng/ml).

In certain embodiments, compositions disclosed herein for culturing APS cells to generate DE cells according to methods of the present disclosure can include a base medium with at least one Activin A modulating agent (and at least one BMP modulator). In some embodiments, compositions disclosed herein for culturing APS cells to generate DE cells according to methods of the present disclosure can include a base medium with at least one Activin A inhibitor (and at least one BMP modulator) and; optionally, include low protein concentration levels as disclosed herein. Non-limiting examples of Activin A inhibitors include A83-01, RepSox, D4476, Ly364947, R268712, SD208, SB505124, SM16, Galunisertib, SB525334, and SB431542, and the like. In some embodiments, compositions disclosed herein for culturing APS cells to generate DE cells according to methods of the present disclosure can include a base medium with at least one Activin A inhibitor (and at least one BMP modulator) at a concentration of at least about 5 ng/ml to at least about 2 μg/ml, for example, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, at least about 1 μg/ml, at least about 1.5 μg/ml, or at least about at least about 2 μg/ml. In some embodiments, compositions disclosed herein for culturing APS cells to generate DE cells according to methods of the present disclosure can include a base medium with at least one Activin A inhibitor at a concentration of about 0.5 μg/ml to about 2 μg/ml (e.g., about 0.5 μg/ml to about 2.0 μg/ml or about 1 μg/ml).

In certain embodiments, compositions disclosed herein for culturing APS cells to generate DE cells according to methods of the present disclosure can be a base medium with a an enriched concentration of protein as disclosed herein and at least one BMP modulating agent, at least one Activin A modulating agent, or a combination thereof. In some embodiments, compositions disclosed herein for culturing APS cells to generate DE cells according to methods of the present disclosure can include a base medium with an enriched concentration of protein and at least one BMP inhibitor, at least one Activin A inhibitor, or a combination thereof. In some embodiments, compositions disclosed herein for culturing APS cells to generate DE cells according to methods of the present disclosure can include a base medium with an enriched concentration of protein, at least one BMP inhibitor, and at least one Activin A inhibitor.

In some embodiments, compositions disclosed herein for culturing APS cells to generate DE cells according to methods of the present disclosure can include a base medium with at least one Activin A activator. In some embodiments, compositions disclosed herein for culturing APS cells to generate DE cells according to methods of the present disclosure can be a base medium with at least one Activin A activator at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, for example. In some embodiments, compositions disclosed herein for culturing APS cells to generate DE cells according to methods of the present disclosure can be a base medium with at least one Activin A activator at a concentration of about 5 ng/ml to about 200 ng/ml (e.g., about 10 ng/ml to about 75 ng/ml or about 15 ng/ml to about 50 ng/ml). In some embodiments, compositions disclosed herein for culturing APS cells to generate DE cells according to methods of the present disclosure can be a base medium with at least one Activin A activator at a concentration of about 5 ng/ml, about 10 ng/ml, about 25 ng/ml, about 50 ng/ml, about 75 ng/ml, about 100 ng/ml, about 125 ng/ml, about 150 ng/ml, about 175 ng/ml, or about 200 ng/ml.

In certain embodiments, compositions disclosed herein for culturing APS cells to generate DE cells can be provided to the cell culture after the previous culture medium is removed or washed away, as appropriate. In accordance with these embodiments, compositions disclosed herein for culturing APS cells to generate DE cells can be provided to an APS cell culture after the previous culture medium is removed with or without a wash step before introduction of the fresh media.

In certain embodiments, compositions disclosed herein for culturing APS cells to generate DE cells can be provided to an APS cell culture and incubated for a period of time suitable for APS cells to differentiate into DE cells. In some embodiments, compositions disclosed herein for culturing APS cells to generate DE cells can be provided to an APS cell culture and incubated for a time period suitable for at least about 70% to at least about 99% or 100% of the APS cells to differentiate into DE cells. In some embodiments, compositions disclosed herein for culturing APS cells to generate DE cells can be provided to an APS cell culture and incubated for a time period suitable for about 70%, about 75%, about 80%, about 85%, about 90%, about 95%, or about 99% or 100% of the APS cells to differentiate into DE cells. In some embodiments, compositions disclosed herein for culturing APS cells to generate DE cells can be provided to an APS cell culture and incubated for a period of about 12 hours to about 5 days. In some embodiments, compositions disclosed herein for culturing APS cells to generate DE cells can be provided to the cell culture and incubated for a period of less than about 1 day, about 1 day, about 2 days, about 3 days, about 4 days, or about 5 days.

### Stages 3 and 4—Culturing DE Cells to Generate AFE Cells and Culturing a FE Cells to Generate VPE Cells or Culturing DE Cells to Generate PE Cells and Culturing PE Cells to Generate VPE Cells.

In certain embodiments, compositions disclosed herein can be used to provide conditions for culturing DE cells to generate PE and/or AFE cells and/or for culturing PE and/or AFE cells to generate VPE cells according to methods of the present disclosure. In accordance with these embodiments, compositions disclosed herein can include a base medium with a high concentration of protein (e.g., a “protein rich medium”). In some embodiments, compositions disclosed herein can be used to provide conditions for culturing DE cells to generate AFE cells and/or for culturing AFE cells to generate VPE cells according to methods of the present disclosure can include a base medium with a high protein concentrations of at least about 1.0% (w/v) or more. In some embodiments, a base medium with high protein concentrations for use herein can have protein concentrations of at least 0.5% (w/v) or more, or about 1.0% (w/v) or more, or about 2.5% (w/v) or more, or about 5.0% (w/v) or more, or about 7.5% (w/v) or more, or about 10.0% (w/v) or more, or about 15.0% (w/v) or more, or about 20% (w/v) or more, or about 25% or more, up to about 30% (w/v). In some embodiments, a base medium with high protein concentrations for use herein can have a protein concentration ranging from at least about 1.0% (w/v) to about 30% (w/v) protein. In accordance with these embodiments, protein of use to supplement medias disclosed herein can be derived from any source. In certain embodiments, the protein can be supplemented using serum, for example, human serum albumin (HSA), bovine serum albumin (BSA), fetal bovine serum (FBS), or other serum or other protein source used in culture medias.

In some embodiments, media can be washed away from differentiated cells disclosed herein and the washed differentiated cells can be used in a pharmaceutical composition. In certain embodiments, cells can be harvested and stored in a buffer for prolonged storage and transport at reduced temperatures for later use. In other embodiments, differentiated cells disclosed herein can be quick frozen and stored for later use in therapeutics or for culturing.

In certain embodiments, compositions disclosed herein for culturing DE cells to generate AFE cells and/or for culturing AFE cells to generate VPE cells according to methods of the present disclosure can include a base medium with at least one bone morphogenetic protein (BMP) modulating agent. In some embodiments, compositions disclosed herein for culturing DE cells to generate AFE cells and/or for culturing AFE cells to generate VPE cells according to methods of the present disclosure can include a base medium with at least one BMP activator. In certain embodiments, these culturing compositions can no longer include a BMP pathway inhibiting agent. Non-limiting examples of BMP activators include, but are not limited to, BMP4, SB4, and the like. In some embodiments, compositions disclosed herein for culturing DE cells to generate AFE cells and/or for culturing AFE cells to generate VPE cells according to methods of the present disclosure can include a protein enriched media of at least about 0.5% % (w/v) protein or more. In some embodiments, a base medium with high protein concentrations for use herein can have protein concentrations of at least about 0.5% (w/v) or more, or about 1.0% (w/v) or more, or about 2.5% (w/v) or more, or about 5.0% (w/v) or more, or about 7.5% (w/v) or more, or about 10.0% (w/v) or more, or about 15.0% (w/v) or more, or about 20% (w/v) or more, or 25% or more, up to about 30% (w/v) and at least one BMP activator. The BMP activator concentration can be at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In some embodiments, compositions disclosed herein for culturing DE cells to generate AFE cells and/or for culturing AFE cells to generate VPE cells according to methods of the present disclosure can include a protein enriched media with at least one BMP activator at a concentration of about 1 ng/ml to about 100 ng/ml (e.g., about 10 ng/ml to about 75 ng/ml or about 15 ng/ml to about 50 ng/ml). In some embodiments, compositions disclosed herein for culturing DE cells to generate AFE cells and/or for culturing AFE cells to generate VPE cells according to methods of the present disclosure can include a protein enriched media with at least one BMP activator at a concentration of about 1.0 ng/ml to about 150 ng/ml or about 1 ng/ml, about 5 ng/ml, about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, or about 100 ng/ml.

In certain embodiments, compositions disclosed herein for culturing DE cells to generate AFE cells and/or for culturing AFE cells to generate VPE cells according to methods of the present disclosure can include a media with an enriched protein concentrations of at least 0.5% (w/v) to about 30% (w/v) or about 0.5% to about 20% (w/v) and at least one fibroblast growth factor receptor (FGFR) activating agent (and at least one BMP activating agent). As used herein, “a FGFR activating agent” can refer to any chemical (e.g., small molecule or other chemical agent), compound, polypeptide, protein or fragment thereof, polynucleotide (DNA or RNA), or other agent that activates an FGFR and/or an FGFR signaling pathway. In some embodiments, compositions disclosed herein for culturing DE cells to generate AFE cells and/or for culturing AFE cells to generate VPE cells according to methods of the present disclosure can be a media with enriched protein concentrations of at least 0.5% (w/v) and at least one FGFR activator (an at least one BMP activator). In some embodiments, compositions disclosed herein for culturing DE cells to generate AFE cells and/or for culturing AFE cells to generate VPE cells according to methods of the present disclosure can be a base medium with at least one FGFR3 activator. Non-limiting examples of FGFR3 activators suitable for use herein can include a fibroblast growth factor (FGF), such as FGF8, FGF1, FGF2, FGF9, or a variant thereof, Heparin, and the like.

In some embodiments, compositions disclosed herein for culturing DE cells to generate AFE cells and/or for culturing AFE cells to generate VPE cells according to methods of the present disclosure can be a media with a high protein concentration of at least 0.5% (w/v) and at least one FGFR3 activator (e.g., FGF8, FGF1, FGF2, FGF9, or a variant thereof). In accordance with these embodiments, compositions disclosed herein for culturing DE cells to generate AFE cells and/or for culturing AFE cells to generate VPE cells according to methods of the present disclosure can be a media with a high protein concentration of at least 0.5% (w/v) and at least one FGFR3 activator (e.g., FGF8, FGF1, FGF2, FGF9, or a variant thereof) at a concentration of at least about 5.0 ng/ml to about 150 ng/ml, 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml (and at least one BMP activator). In some embodiments, the FGF can be present in the cell culture medium at a concentration ranging of about 10 ng/ml to about 100 ng/ml (e.g., about 20 ng/ml to about 100 ng/ml, or about 30 ng/ml to about 100 ng/ml). In some embodiments, the FGF can be present in the cell culture medium at a concentration of about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, or about 100 ng/ml or about 150 ng/ml.

In some embodiments, compositions disclosed herein for culturing DE cells to generate AFE cells and/or for culturing AFE cells to generate VPE cells according to methods of the present disclosure can include a media having high protein concentrations of at least 0.5% (w/v) and that includes heparin (and optionally, at least one of at least one BMP activator and at least one FGFR3 activator). In accordance with these embodiments, compositions disclosed herein for culturing DE cells to generate AFE cells and/or for culturing AFE cells to generate VPE cells according to methods of the present disclosure can include a high protein media that includes heparin at a concentration ranging from about 1.0 μg/ml to about 100 μg/ml. In certain embodiments, the concentration of heparin in the culture medium is about 1 μg/ml, about 2 μg/ml, about 4 μg/ml, about 6 μg/ml, about 8 μg/ml, about 10 μg/ml, about 12 μg/ml, about 14 μg/ml, about 16 μg/ml, about 18 μg/ml, or about 20 μg/ml.

In certain embodiments, compositions disclosed herein for culturing DE cells to generate AFE cells and/or for culturing AFE cells to generate VPE cells according to methods of the present disclosure can include a media having an enriched protein concentration of at least 0.5% (w/v) protein and at least one Retinoic Acid signaling modulating agent (and optionally, heparin, and at least one BMP activator and at least one FGFR3 activator). As used herein, “a Retinoic Acid signaling modulating agent” can refer to any chemical (e.g., small molecule or other chemical agent), compound, polypeptide, protein, or fragment thereof, polynucleotide (DNA or RNA), or other agent that activates Retinoic Acid, a Retinoic Acid receptor, and/or a Retinoic Acid signaling pathway. In some embodiments, compositions disclosed herein for culturing DE cells to generate AFE cells and/or for culturing AFE cells to generate VPE cells according to methods of the present disclosure can include a media having a high protein concentration of at least 0.5% (w/v) protein and at least one Retinoic Acid signaling activating agent (and optionally, heparin, and at least one BMP activator and at least one FGFR3 activator). Non-limiting examples of Retinoic Acid signaling activators include, but are not limited to, Vitamin A, TTNPB, AC261066, SR1078, SR221, BMS493, Fenretinide, AM580, Adapalene, Ch55, and the like. In some embodiments, compositions disclosed herein for culturing DE cells to generate AFE cells and/or for culturing AFE cells to generate VPE cells according to methods of the present disclosure can include a media having a high protein concentration of at least 0.5% (w/v) protein and at least one Retinoic Acid signal activating agent (and optionally, heparin, and at least one BMP activator and at least one FGFR3 activator) where the Retinoic acid signal activating agent can be present in compositions disclosed herein at a concentration ranging from about 0.1 nM/ml to about 3,000 nM/ml; about 1.0 nM/ml to about 2,500 nM/ml, about 1.5 nM/ml to about 2,000 nM/ml or any concentration in between in order to obtain the desired signaling response. In some embodiments, a Retinoic Acid signaling activator can be present in compositions disclosed herein at a concentration of about 0.1 nM, about 0.5 nM, about 1.0 nM, about 2.5 nM, about 5.0 nM, about 10 nM, about 15 nM, about 20 nM, about 30 nM, about 40 nM, about 50 nM, about 60 nM, about 70 nM, about 80 nM, about 90 nM, about 100 nM, about 125 nM, about 150 nM, about 175 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1.0 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2 μM, about 2.3 μM, about 2.4 μM, about 2.5 μM, about 2.6 μM, about 2.7 μM, about 2.8 μM, about 2.9 μM, or about 3.0 μM.

In certain embodiments, compositions disclosed herein for culturing DE cells to generate AFE and/or PE cells and/or for culturing AFE and/or PE cells to generate VPE cells according to methods of the present disclosure can include a media having a high concentration of protein and at least one of, at least one BMP activating agent, at least one FGFR3 activating agent, at least one FGFR2 activating agent, at least one NOTCH activating agent or a combination thereof. In certain embodiments, compositions disclosed herein for culturing DE cells to generate AFE and/or PE cells and/or for culturing AFE and/or PE cells to generate VPE cells according to methods herein can include media having a high concentration of protein and at least one of at least one BMP activating agent, at least one FGFR3 activating agent, at least one FGFR2 activating agent, at least one NOTCH activating agent, at least one agent that activates Retinoic acid receptor signaling or a combination thereof. In certain embodiments, compositions disclosed herein for culturing DE cells to generate AFE and/or PE cells and/or for culturing AFE and/or PE cells to generate VPE cells according to methods of the present disclosure can be a media having a high concentration of protein and at least one BMP activating agent, at least one FGFR3 activating agent, at least one FGFR2 activating agent, at least one NOTCH activating agent, at least one agent that activates Retinoic acid receptor signaling, one sonic hedgehog signaling activating agent or a combination thereof. In certain embodiments, compositions disclosed herein for culturing DE cells to generate AFE and/or PE cells and/or for culturing AFE and/or PE cells to generate VPE cells according to methods of the present disclosure can be a media having a high concentration of protein and at least one BMP activating agent, at least one agent that activates Retinoic acid receptor signaling, at least one inhibitor of TGF-β receptor signaling, and at least one sonic hedgehog signaling activating agent, at least one VEGF receptor activating agent or a combination thereof.

In certain embodiments, compositions disclosed herein for culturing DE cells to generate AFE cells and/or for culturing AFE cells to generate VPE cells according to methods of the present disclosure can be a media having a high concentration of protein and at least one BMP modulating agent, at least one FGFR3 activating agent, at least one a Retinoic Acid signaling activating agent or a combination thereof. In some embodiments, compositions disclosed herein for culturing DE cells to generate AFE cells and/or for culturing AFE cells to generate VPE cells according to methods of the present disclosure can be a composition or media with a high concentration of protein and at least one BMP activator, at least one FGFR3 activator, at least one a Retinoic Acid signaling activator, or a combination thereof. In some embodiments, compositions disclosed herein for culturing DE cells to generate AFE cells and/or for culturing AFE cells to generate VPE cells according to methods of the present disclosure can include a composition or media with a high concentration of protein (e.g., enriched concentration) that includes at least one of at least one BMP activator, at least one FGFR3 activator, at least one a Retinoic Acid signaling activator, and heparin. In certain embodiments, these media compositions can further include at least one Wnt pathway signaling activator and/or NOTCH pathway signaling activator.

In certain embodiments, compositions disclosed herein for culturing DE cells to generate AFE cells and/or for culturing AFE cells to generate VPE cells do not include a transforming growth factor-b (TGF-β) modulating agent. In certain embodiments, compositions disclosed herein for culturing DE cells to generate AFE cells and/or for culturing AFE cells to generate VPE cells do not include a TGF-β inhibitor.

In certain embodiments, compositions disclosed herein for culturing DE cells to generate AFE cells and/or for culturing AFE cells to generate VPE cells can replace a cell culture media after the previous culture medium is removed and optionally, washed to remove remaining agents. In accordance with these embodiments, compositions disclosed herein for culturing DE cells to generate AFE cells and/or for culturing AFE cells to generate VPE cells can be provided to the cell culture after the previous culture medium is removed with or without a wash step before application.

In certain embodiments, compositions disclosed herein for culturing DE cells to generate AFE cells and/or for culturing AFE cells to generate VPE cells can be provided to the cell culture and incubated for a period of time suitable for DE cells to differentiate into AFE cells. In some embodiments, compositions disclosed herein for culturing DE cells to generate AFE cells and/or for culturing AFE cells to generate VPE cells can be provided to the cell culture and incubated for a period of time suitable for at least about 5%, about 10%, about 15%, about 20%, about 25%, about 30%, about 35%, about 40%, about 45%, about 50%, about 55%, about 60%, about 65%, about 70% to about 99% or about 100% of the DE cells differentiate into AFE cells. In some embodiments, compositions disclosed herein for culturing DE cells to generate AFE cells and/or for culturing AFE cells to generate VPE cells can be provided to the cell culture and incubated for a period of time suitable for about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% or 100% of the DE cells to differentiate into AFE cells.

In certain embodiments, compositions disclosed herein for culturing DE cells to generate AFE cells and/or for culturing AFE cells to generate VPE cells can be provided to the cell culture and incubated for a time period suitable for AFE cells to differentiate into VPE cells. In some embodiments, compositions disclosed herein for culturing DE cells to generate AFE cells and/or for culturing AFE cells to generate VPE cells can be provided to the cell culture and incubated for a period of time suitable for at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% or 100% of the AFE cells to differentiate into VPE cells. In some embodiments, compositions disclosed herein for culturing compositions disclosed herein for culturing DE cells to generate AFE cells and/or for culturing AFE cells to generate VPE cells can be provided to the cell culture and incubated for a time period suitable for about 70%, about 75%, about 80%, about 85%, about 90%, about 95%, or about 99% or 100% of the AFE cells to differentiate into VPE cells.

In some embodiments, culturing DE cells to generate AFE cells and/or for culturing AFE cells to generate VPE cells can include culturing the cells for a period of about 1 day to about 9 days in medias with supplements as indicated herein. In some embodiments, compositions for culturing DE cells to generate AFE cells and/or for culturing AFE cells to generate VPE cells can include, but is not limited to, culturing cells in compositions disclosed herein for a period of about 12 hours, to about 1 day, to about 2 days, to about 3 days, to about 4 days, or about 5 days to about 9 days or more.

In some embodiments, culturing DE cells to generate AFE cells and/or for culturing AFE cells to generate VPE cells can include culturing the cells for a period of about 1 day to about 9 days in medias with supplements as indicated herein. In some embodiments, compositions for culturing DE cells to generate AFE cells and/or for culturing AFE cells to generate VPE cells can include, but is not limited to, culturing cells in compositions disclosed herein for a period of about 12 hours, to about 1 day, to about 2 days, to about 3 days, to about 4 days, or about 5 days to about 9 days or more. In certain embodiments, compositions disclosed herein for culturing DE cells to generate AFE cells and/or for culturing AFE cells to generate VPE cells according to methods of the present disclosure can be a media having a high concentration of protein and at least one at least one NOTCH pathway signaling activator, at least one CD40 pathway signaling activator, and/or at least one RANK signaling pathway activator. In accordance with these embodiments, compositions disclosed herein having at least one NOTCH pathway activator can include, but are not limited to, Yhhu 3792, DLL1, DLL3, DLL4, Jagged 1, and Jagged 2. In certain embodiments, compositions disclosed herein can include or further include at least one CD40 pathway activator. In accordance with these embodiments, the at least one CD40 pathway activator can include, but are not limited to, CD40 ligand, multimeric CD40 ligand, CD40 ligand and a crosslinker, and an anti-CD40 antibody with an antibody crosslinker. In some embodiments, compositions disclosed herein can include or further include at least one RANK pathway activator. In accordance with these embodiments, the at least one RANK pathway activator can include. but are not limited to, soluble RANK ligand, RANK ligand, multimeric RANK ligand, RANK ligand and a crosslinker, and an anti-RANK antibody with an antibody crosslinker. In other embodiments, compositions disclosed herein can further include at least one Ghrelin receptor pathway activator. In accordance with the embodiments, the at least one Ghrelin receptor pathway activator can include, but are not limited to, Ghrelin, MK 0677, Tabimorelin hemifumerate, and L-692,585. In certain embodiments, compositions disclosed herein can include at least one NOTCH pathway activator and at least one of, at least one CD40 pathway activator and at least one RANK pathway activator. In accordance with these embodiments, compositions disclosed herein can include a protein enriched culture medium (e.g., MEM alpha media).

### Stage 5—Culturing VPE or TPPE Cells to Generate Thymic Cells

In certain embodiments, compositions disclosed herein can be used to provide conditions for culturing VPE and/or TPPE cells to generate thymic cells (TEC and or TEP cells) according to methods of the present disclosure. In accordance with these embodiments, compositions disclosed herein can include a base medium having a high or enriched concentration of protein (e.g., a “protein enriched medium”). In some embodiments, high protein concentrations can include at least about 0.5% (w/v) to about 30% (w/v). In some embodiments, a base medium with high protein concentrations for use herein can include protein concentrations of at least 0.5% (w/v) to about 1.0% (w/v), or about 2.0% (w/v), or about 3.0% (w/v), or about 4.0% (w/v), or about 5.0% (w/v), or about 10% (w/v), or about 20% (w/v), or about 30% (w/v) protein or any concentration between at least 0.5% (w/v) and 30.0% (w/v). In some embodiments, a base medium with enriched protein concentrations of use as compositions disclosed herein can include a protein concentration ranging from at least 0.5% (w/v) to about 30% (w/v) (e.g., about 1.0% (w/v), about 5.0% (w/v), about 10% (w/v), about 15% (w/v), about 20% (w/v), about 25% (w/v), about 30% (w/v)).

In some embodiments, compositions disclosed herein for culturing VPE and/or TPPE cells to generate thymic cells according to methods of the present disclosure can include a protein rich media and at least one BMP modulating agent. In some embodiments, compositions disclosed herein for culturing VPE and/or TPPE cells to generate thymic cells according to methods of the present disclosure can include a protein rich media and at least one BMP inhibitor. In some embodiments, compositions disclosed herein for culturing VPE and/or TPPE cells to generate thymic cells according to methods of the present disclosure can include a protein rich media and at least one BMP inhibitor at a concentration of at least about 5 ng/ml to about 1,000 ng/ml or at least about 10 ng/ml, at least about 25 ng/ml, at least about 50 ng/ml, at least about 75 ng/ml, at least about 100 ng/ml, at least about 200 ng/ml, at least about 250 ng/ml, at least about 300 ng/ml, at least about 400 ng/ml, at least about 500 ng/ml, or at least about 1000 ng/ml or any concentration between 10 ng/ml and 1,000 ng/ml, for example, at a concentration of at least 10 ng/ml, at least 25 ng/ml, at least 50 ng/ml, at least 75 ng/ml, at least 100 ng/ml, at least 200 ng/ml, at least about 250 ng/ml, at least 300 ng/ml, at least 400 ng/ml, at least 500 ng/ml, or at least 1000 ng/ml. In some embodiments, compositions disclosed herein for culturing VPE and/or TPPE cells to generate thymic cells according to methods disclosed herein can include a protein enriched media and at least one least one BMP inhibitor at a concentration of about 5 ng/ml to about 300 ng/ml (e.g., about 10 ng/ml to about 250 ng/ml or about 15 ng/ml to about 100 ng/ml).

In some embodiments, compositions disclosed herein for culturing VPE and/or TPPE cells to generate thymic cells according to methods of the present disclosure can include a protein enriched media with at least one Activin A modulating agent. In some embodiments, compositions disclosed herein for culturing VPE and/or TPPE cells to generate thymic cells according to methods of the present disclosure can include a protein-rich media with at least one Activin A activator. In some embodiments, compositions disclosed herein for culturing VPE and/or TPPE cells to generate thymic cells according to methods of the present disclosure can include a protein-rich media having at least 0.5% (w/v) protein up to about 30% (w/v) protein and at least one Activin A activator at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, for example. In some embodiments, compositions disclosed herein for culturing VPE and/or TPPE cells to generate thymic cells according to methods of the present disclosure can include a protein-rich media having at least 1% (w/v) protein up to about 30% (w/v) protein and at least one Activin A activator at a concentration of about 5 ng/ml to about 200 ng/ml (e.g., about 10 ng/ml to about 100 ng/ml or about 15 ng/ml to about 50 ng/ml).

In certain embodiments, compositions disclosed herein for culturing VPE and/or TPPE cells to generate thymic cells according to methods of the present disclosure can include a protein-rich media having at least 0.5% (w/v) protein up to about 30% (w/v) protein and at least one Retinoic Acid signaling modulating agent (and optionally at least one Activin A activator and BMP inhibitor). In some embodiments, compositions disclosed herein for culturing VPE and/or TPPE cells to generate thymic cells according to methods of the present disclosure can include a protein-rich media having at least 0.5% (w/v) protein up to about 30% (w/v) protein and at least one Retinoic Acid signaling activator. In some embodiments, compositions disclosed herein for culturing VPE and/or TPPE cells to generate thymic cells according to methods of the present disclosure can include a protein-rich media having at least 0.5% (w/v) protein up to about 30% (w/v) protein and at least one Retinoic Acid signaling activator at a concentration ranging from about 0.1 nM/ml to about 3,000 nM/ml; about 1.0 nM/ml to about 2,500 nM/ml, about 1.5 nM/ml to about 2,000 nM/ml or any concentration in between in order to obtain the desired signaling response. In some embodiments, a Retinoic Acid signaling activator can be present in compositions disclosed herein at a concentration of about 0.1 nM, about 0.5 nM, about 1.0 nM, about 2.5 nM, about 5.0 nM, about 10 nM, about 15 nM, about 20 nM, about 30 nM, about 40 nM, about 50 nM, about 60 nM, about 70 nM, about 80 nM, about 90 nM, about 100 nM, about 125 nM, about 150 nM, about 175 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1.0 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2 μM, about 2.3 μM, about 2.4 μM, about 2.5 μM, about 2.6 μM, about 2.7 μM, about 2.8 μM, about 2.9 μM, or about 3.0 μM. In some embodiments, a Retinoic Acid signaling activator can be present in the cell culture medium at a concentration ranging from about 0.1 nM to about 3.0 μM (e.g., about 0.5 nM to about 2.5 μM or about 1 nM to about 2 μM) and further include at least one Activin A activator and BMP inhibitor.

In certain embodiments and further to paragraphs [0038]-[0077] above, concentration of agents disclosed herein of use to supplement medias (e.g., low protein or enriched protein medias) disclosed herein can be from about 1 nM to about 10 μM in final concentrations. In some embodiments, concentration of agents disclosed herein of use to supplement medias disclosed herein can be about 5 to about 15 (e.g., about 9 nM) for a small molecule and about 0.5 μM to about 5 μM (e.g., about 2 μM) for a bio-molecules or other agents.

In some embodiments, compositions disclosed herein for culturing VPE and/or TPPE cells to generate thymic cells according to methods of the present disclosure can include a protein-rich media having at least 0.5% (w/v) protein up to about 30.0% (w/v) protein and further includes heparin (and optionally at least one: at least one Activin A activator, at least one BMP inhibitor and at least one retinoic acid receptor activator). In accordance with these embodiments, compositions disclosed herein for culturing VPE and/or TPPE cells to generate thymic cells according to methods of the present disclosure can include a protein-rich media having at least 0.5% (w/v) protein up to about 30.0% (w/v) protein and further includes heparin at a concentration ranging from about 1 μg/ml to about 100 μg/ml. In certain embodiments, the concentration of heparin in the culture medium is about 1 μg/ml, about 2 μg/ml, about 4 μg/ml, about 6 μg/ml, about 8 μg/ml, about 10 μg/ml, about 12 μg/ml, about 14 μg/ml, about 16 μg/ml, about 18 μg/ml, or about 20 μg/ml.

In certain embodiments, compositions disclosed herein for culturing VPE and/or TPPE cells to generate thymic cells according to methods of the present disclosure can include a protein-rich media having at least 0.5% (w/v) protein up to about 30.0% (w/v) protein and further includes at least one fibroblast growth factor receptor (FGFR) modulating agent. In some embodiments, compositions disclosed herein for culturing VPE and/or TPPE cells to generate thymic cells according to methods of the present disclosure can include a protein-rich media having at least 0.5% (w/v) protein up to about 30.0% (w/v) protein and further includes at least one FGFR activator (and optionally at least one: at least one Activin A activator, at least one BMP inhibitor and at least one retinoic acid receptor activator and heparin). In some embodiments, compositions disclosed herein for culturing VPE and/or TPPE cells to generate thymic cells according to methods of the present disclosure can include a protein-rich media having at least 0.5% (w/v) protein up to about 30.0% (w/v) protein and further includes at least one FGFR activator (and optionally at least one: at least one Activin A activator, at least one BM P inhibitor and at least one retinoic acid receptor activator and heparin) wherein the FGFR activator includes, but is not limited to, at least one FGFR1 activator, at least one FGFR2 activator, or a combination thereof. Non-limiting examples of FGFR1 and FGFR2 activators suitable for use herein can include a fibroblast growth factor (FGF), such as FGF10, FGF3, FGF4, FGF7, and FGF22, or a variant thereof. In some embodiments, compositions disclosed herein for culturing VPE and/or TPPE cells to generate thymic cells according to methods of the present disclosure can include a protein-rich media having at least 0.5% (w/v) protein up to about 30% (w/v) protein and further includes at least one FGFR activator (and optionally at least one: at least one Activin A activator, at least one BMP inhibitor and at least one retinoic acid receptor activator and heparin) including, but not limited to, FGF (e.g., FGF10, FGF3, FGF4, FGF7, and FGF22, or a variant thereof). In accordance with these embodiments, compositions disclosed herein for culturing VPE and/or TPPE cells to generate thymic cells according to methods of the present disclosure can include a protein-rich media having at least 0.5% (w/v) protein up to about 30% (w/v) protein and further includes at least one FGFR activator (and optionally at least one: at least one Activin A activator, at least one BMP inhibitor and at least one retinoic acid receptor activator and heparin) including, but not limited to, FGF (e.g., FGF10, FGF3, FGF4, FGF7, and FGF22, or a variant thereof) at a concentration of at least about 0.5 ng/ml, at least about 1.0 ng/ml, at least about 1.5 ng/ml, at least about 2.0 ng/ml, at least about 2.5 ng/ml, at least about 3.0 ng/ml, at least about 3.5 ng/ml, at least about 4.0 ng/ml, at least about 4.5 ng/ml, at least about 5.0 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In some embodiments, the FGF can be present in the cell culture medium at a concentration ranging from about 0.5 ng/ml to about 100 ng/ml (e.g., about 1.0 ng/ml to about 100 ng/ml, or about 2.0 ng/ml to about 100 ng/ml).

In certain embodiments, compositions disclosed herein for culturing VPE and/or TPPE cells to generate thymic cells according to methods of the present disclosure can include a base medium with at least one hedgehog signal modulating agent, including sonic hedgehog (Shh) As used herein, “a Shh modulating agent” can refer to any chemical (e.g. small molecule or other chemical agent), compound, polypeptide, protein or fragment thereof, polynucleotide (DNA or RNA), or other agent that modulates hedgehog and/or Shh and/or a Shh signaling pathway. In some embodiments, compositions disclosed herein for culturing VPE and/or TPPE cells to generate thymic cells according to methods of the present disclosure can include a protein-rich media having at least 1.0% (w/v) protein up to about 30.0% (w/v) protein and further includes at least one Shh inhibitor (and optionally at least one: at least one Activin A activator, at least one BMP inhibitor and at least one retinoic acid receptor activator, at least one FGFR2 activator and heparin). Non-limiting Shh inhibitors suitable for uses disclosed herein can include SANT1, SANT2, U1866A, Dynapyrazole a, Dynarrestin, Cyclopamine, HIP1, GANT58, AY9944 dihydrochloride, RU-SKI 43 hydrochloride, and the like. In some embodiments, compositions disclosed herein for culturing VPE and/or TPPE cells to generate thymic cells according to methods of the present disclosure can include a protein-rich media having at least 1.0% (w/v) protein up to about 30.0% (w/v) protein and further includes at least one Shh inhibitor (and optionally at least one: at least one Activin A activator, at least one BMP inhibitor and at least one retinoic acid receptor activator, at least one FGFR2 activator and heparin) in the medium at a concentration of at least about 0.5 nM, about 1.0 nM, about 2.0 nM, about 3.0 nM, about 4.0 nM, about 5.0 nM, about 10 nM, about 25 nM, about 50 nM, about 75 nM, about 100 nM, about 125 nM, about 150 nM, about 175 nM, about 200 nM, about 225 nM, about 250 nM, about 275 nM, about 300 nM, about 325 nM, about 350 nM, about 375 nM, about 400 nM, about 425 nM, about 450 nM, about 475 nM, about 500 nM, about 525 nM, about 550 nM, about 575 nM, about 600 nM, about 625 nM, about 650 nM, about 675 nM, about 700 nM, about 725 nM, about 750 nM, about 775 nM, about 800 nM, about 825 nM, about 850 nM, about 875 nM, about 900 nM, about 925 nM, about 950 nM, about 975 nM, or about 1000 nM.

In certain embodiments, compositions disclosed herein for culturing VPE and/or TPPE cells to generate thymic cells according to methods of the present disclosure can include a base medium having an enriched protein concentration and at least one of, at least one NOTCH pathway signaling activator, at least one CD40 pathway signaling activator, and/or at least one RANK signaling pathway activator. In accordance with these embodiments, compositions disclosed herein having at least one NOTCH pathway activator can include, but are not limited to, Yhhu 3792, DLL1, DLL3, DLL4, Jagged 1, and Jagged 2. In certain embodiments, compositions disclosed herein can include or further include at least one CD40 pathway activator. In accordance with these embodiments, the at least one CD40 pathway activator can include, but are not limited to, CD40 ligand, multimeric CD40 ligand, CD40 ligand and a crosslinker, and an anti-CD40 antibody with an antibody crosslinker. In some embodiments, compositions disclosed herein can include or further include at least one RANK pathway activator. In accordance with these embodiments, the at least one RANK pathway activator can include, but are not limited to, soluble RANK ligand, RANK ligand, multimeric RANK ligand, RANK ligand and a crosslinker, and an anti-RANK antibody with an antibody crosslinker. In other embodiments, compositions disclosed herein can further include at least one Ghrelin receptor pathway activator. In accordance with the embodiments, the at least one Ghrelin receptor pathway activator can include, but are not limited to, Ghrelin, MK 0677, Tabimorelin hemifumerate, and L-692,585. In certain embodiments, compositions disclosed herein can include at least one NOTCH pathway activator and at least one of, at least one CD40 pathway activator and at least one RANK pathway activator. In accordance with these embodiments, compositions disclosed herein can include a protein enriched culture medium (e.g., MEM alpha media).

In certain embodiments, compositions disclosed herein for culturing VPE and/or TPPE cells to generate thymic cells according to methods of the present disclosure can include a protein-enriched media having at least 0.5% (w/v) protein up to about 30.0% (w/v) protein and further includes at least one Epidermal Growth Factor (EGF) (and optionally at least one: at least one Activin A activator, at least one BMP inhibitor and at least one retinoic acid receptor activator, at least one FGFR2 activator, at least one Shh inhibitor and heparin). In some embodiments, compositions disclosed herein for culturing VPE and/or TPPE cells to generate thymic cells according to methods of the present disclosure can include a protein-rich media having at least 0.5% (w/v) protein up to about 30.0% (w/v) protein and further includes at least one Epidermal Growth Factor (EGF) (and optionally at least one: at least one Activin A activator, at least one BMP inhibitor and at least one retinoic acid receptor activator, at least one FGFR2 activator, at least one Shh inhibitor and heparin) where the EGF is present at concentrations ranging from about 0.1 ng/ml to about 30.0 ng/ml. In some embodiments, compositions disclosed herein for culturing VPE and/or TPPE cells to generate thymic cells according to methods of the present disclosure can include a protein-rich media having at least 0.5% (w/v) protein up to about 30.0% (w/v) protein and further includes at least one Epidermal Growth Factor (EGF) (and optionally at least one: at least one Activin A activator, at least one BMP inhibitor and at least one retinoic acid receptor activator, at least one FGFR2 activator, at least one Shh inhibitor and heparin) having a concentration of EGF of about 0.1 ng/ml, about 0.5 ng/ml, about 1.0 ng/ml, about 5.0 ng/ml, about 10 ng/ml, about 15 ng/ml, about 20 ng/ml, about 25 ng/ml, about 30 ng/ml, 40 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, about 275 ng/ml, about 300 ng/ml, about 325 ng/ml, about 350 ng/ml, about 375 ng/ml, about 400 ng/ml, about 425 ng/ml, about 450 ng/ml, about 475 ng/ml, or about 500 ng/ml.

In certain embodiments, compositions disclosed herein for culturing VPE and/or TPPE cells to generate thymic cells according to methods of the present disclosure can include a protein enriched media and optionally, include at least one Wnt modulating agent. In some embodiments, compositions disclosed herein for culturing VPE and/or TPPE cells to generate thymic cells according to methods of the present disclosure can be in a protein rich media optionally including at least one Wnt activator. In some embodiments, compositions disclosed herein for culturing VPE and/or TPPE cells to generate thymic cells according to methods of the present disclosure can be a base medium optionally including at least one Wnt activator at a concentration ranging from about 5 ng/ml to about 200 ng/ml (e.g., about 10 ng/ml to about 150 ng/ml or about 15 ng/ml to about 100 ng/ml).

In certain embodiments, compositions disclosed herein for culturing VPE and/or TPPE cells to generate thymic cells according to methods of the present disclosure can include a protein enriched media optionally including at least one NOTCH activating agent. In some embodiments, compositions disclosed herein for culturing VPE and/or TPPE cells to generate thymic cells according to methods of the present disclosure can include a protein enriched media optionally including at least one NOTCH activator. In some embodiments, compositions disclosed herein for culturing VPE and/or TPPE cells to generate thymic cells according to methods of the present disclosure can include a protein rich media optionally include at least on NOTCH activator at a concentration ranging from about 5 ng/ml to about 100 ng/ml (e.g., about 10 ng/ml to about 75 ng/ml or about 15 ng/ml to about 50 ng/ml).

In certain embodiments, compositions disclosed herein for culturing VPE and/or TPPE cells to generate thymic cells according to methods of the present disclosure can include a protein rich media optionally including at least one RANK activating agent. In some embodiments, compositions disclosed herein for culturing VPE and/or TPPE cells to generate thymic cells according to methods of the present disclosure can include a protein rich media optionally including at least one RANK activator. In some embodiments, compositions disclosed herein for culturing VPE and/or TPPE cells to generate thymic cells according to methods of the present disclosure can include a protein rich media optionally include at least on RANK activator at a concentration ranging from about 5 ng/ml to about 100 ng/ml (e.g., about 10 ng/ml to about 75 ng/ml or about 15 ng/nl to about 50 ng/ml).

In certain embodiments, compositions disclosed herein for culturing VPE and/or TPPE cells to generate thymic cells according to methods of the present disclosure can include a protein enriched media optionally including at least one CD40 activating agent. In some embodiments, compositions disclosed herein for culturing VPE and/or TPPE cells to generate thymic cells according to methods of the present disclosure can include a protein rich media optionally including at least one CD40 activator. In some embodiments, compositions disclosed herein for culturing VPE and/or TPPE cells to generate thymic cells according to methods of the present disclosure can include a protein rich media optionally include at least on CD40 activator at a concentration ranging from about 5 ng/ml to about 100 ng/ml (e.g., about 10 ng/ml to about 75 ng/ml or about 15 ng/ml to about 50 ng/ml).

In certain embodiments, compositions disclosed herein for culturing VPE and/or TPPE cells to generate thymic cells according to methods of the present disclosure can include a protein enriched media optionally including at least one Ghrelin receptor signaling activating agent. In some embodiments, compositions disclosed herein for culturing VPE and/or TPPE cells to generate thymic cells according to methods of the present disclosure can include a protein rich media optionally including at least one Ghrelin receptor signaling activator. In some embodiments, compositions disclosed herein for culturing VPE and/or TPPE cells to generate thymic cells according to methods of the present disclosure can include a protein rich media optionally include at least on Ghrelin receptor signaling activator at a concentration ranging from about 5 ng/ml to about 100 ng/ml (e.g., about 10 ng/ml to about 75 ng/ml or about 10 ng/ml to about 30 ng/ml).

In certain embodiments, compositions disclosed herein for culturing VPE and/or TPPE cells to generate thymic cells according to methods of the present disclosure can include a base medium enriched in protein and at least one Activin A modulating agent, at least one BMP modulating agent, at least one FGFR modulating agent, at least one a Retinoic Acid signaling modulating agent, at least one Shh modulating agent, optionally a Wnt activator and/or NOTCH activator or a combination thereof.

In some embodiments, compositions disclosed herein for culturing VPE and/or TPPE cells to generate thymic cells according to methods of the present disclosure can include a base having an enriched protein content and at least one Activin A activator, at least one BMP inhibitor, at least one FGFR activator, at least one a Retinoic Acid signaling activator, at least one Shh inhibitor, or a combination thereof. In some embodiments, compositions disclosed herein for culturing VPE and/or TPPE cells to generate thymic cells according to methods of the present disclosure can be a base medium with a high concentration of protein and includes at least one Activin A activator, at least one BMP inhibitor, at least one FGFR1 activator/FGFR2 activator, at least one a Retinoic Acid signaling activator, at least one Shh inhibitor, heparin and EGF.

In certain embodiments, compositions disclosed herein for culturing VPE and/or TPPE cells to generate thymic cells do not include a transforming growth factor-b (TGF-β) modulating agent. In certain embodiments, compositions disclosed herein for culturing VPE and/or TPPE cells to generate thymic cells do not include a TGF-β inhibitor.

In certain embodiments, compositions disclosed herein for culturing VPE and/or TPPE cells to generate thymic cells can be provided to the cell culture after the previous culture medium is removed. In accordance with these embodiments, compositions disclosed herein for culturing VPE and/or TPPE cells to generate thymic cells can be provided to the cell culture after the previous culture medium is removed without a wash step before application.

In certain embodiments, compositions disclosed herein for culturing VPE and/or TPPE cells to generate thymic cells can be provided to the cell culture for a period of time suitable for VPE and/or TPPE cells to differentiate into thymic cells. In some embodiments, compositions disclosed herein for culturing VPE and/or TPPE cells to generate thymic cells can be provided to the cell culture for a period of time suitable for at least about 70% to at least about 99% of the VPE and/or TPPE cells to differentiate into thymic cells. In some embodiments, compositions disclosed herein for culturing VPE and/or TPPE cells to generate thymic cells can be provided to the cell culture for a period of time suitable for about 70%, about 75%, about 80%, about 85%, about 90%, about 95%, or about 99% of the VPE and/or TPPE cells to differentiate into thymic cells. In some embodiments, compositions disclosed herein for culturing VPE and/or TPPE cells to generate thymic cells can be introduced to the cell culture and incubated a period of about 1 day to about 5 days. In some embodiments, compositions disclosed herein for compositions disclosed herein for culturing VPE and/or TPPE cells to generate thymic cells can be provided to the cell culture and incubated for a period of less than about 1 day, about 1 day, about 2 days, about 3 days, about 4 days, or about 5 days.

In certain embodiments, one or more additional supplements can also be added to any of the compositions described herein to supply the cells with trace elements for improved growth and expansion. In certain embodiments, supplemental trace elements can include, but are not limited to, iron (Fe), copper (Cu), zinc (Zn), rubidium (Rb), selenium (Se), strontium (Sr), molybdenum (Mo), manganese (Mn), lead (Pb), arsenic (As), chromium (Cr), cobalt (Co), vanadium (V), and cadmium (Cd). In certain embodiments, supplemental trace elements can include, but are not limited to, iron (Fe) (hemoglobin), copper (Cu), cobalt (Co) (Vitamin B12), iodine, manganese (Mn) and zinc (Zn) or a combination thereof. In other embodiments, additional supplements can be used compositions used in one or more stages of differentiation for producing thymic cells from PSCs. Such supplements can include, but are not limited to, insulin, transferrin, sodium selenium, and combinations thereof. These components can be included in any known acceptable form. In some embodiments, these agents can be in a salt solution including, but not limited to, Hanks' Balanced Salt Solution® (HBSS), Earle's Salt Solution®, antioxidant supplements, MCDB-201® supplements, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids. In some examples, amino acids for use herein can include, but are not limited to, L-alanine, L-arginine, L-aspartic acid, L-asparagine, L-cysteine, L-cystine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine. In other embodiments,

In other aspects, as needed, antibiotics can be used in cell cultures to mitigate or eliminate bacterial, mycoplasma, and fungal contamination. Typically, antibiotics or anti-mycotic compounds used are mixtures of penicillin/streptomycin, but can also include, but are not limited to, amphotericin (Fungizone®), ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be used in cell cultures and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine. β-mercaptoethanol and other hormones contemplated herein.

Lipids and lipid carriers can also be used to supplement cell culture media, depending on the type of cell and the fate of the differentiated cell. Such lipids and carriers can include, but are not limited to cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

In some embodiments, compositions herein can include a base medium supplemented with an Insulin-Transferrin-Selenium (ITS) supplement. In accordance with these embodiments, ITS can be provided to the medium in concentrations ranging from about 1:10 (v/v) to about 1:10,000 (v/v) or in concentrations greater than about 1:200 (v/v). In some embodiments, the concentration of ITS in the medium can be about 1:1000 (v/v), about 1:900 (v/v), about 1:800 (v/v), about 1:700 (v/v), about 1:600 (v/v), about 1:500 (v/v), about 1:400 (v/v), about 1:300 (v/v), about 1:200 (v/v), about 1:100 (v/v), or about 1:50 (v/v).

In certain embodiments and further to the preceding paragraphs, compositions herein can include a base medium supplemented with cortisol, e.g., hydrocortisone. In accordance with these embodiments, cortisol can be provided to the medium in concentrations ranging from about 0.05 μg/ml to about 5 μg/ml. In some embodiments, the concentration of hydrocortisone, e.g., in the culture medium is about 0.1 μg/ml, 0.2 μg/ml, 0.3 μg/ml, 0.4 μg/ml, 0.5 μg/ml, 0.6 μg/ml, 0.7 μg/ml, 0.8 μg/ml 0.9 μg/ml, or about 1.0 μg/ml. In certain embodiments, a based medium disclosed here can further include serum or other protein supplements or other agents contemplated herein.

In some embodiments, cells of the present disclosure in culture can be maintained either in suspension or attached to a solid support, such as a coated plate or where extracellular matrix components and synthetic or biopolymers are included. Cells can also be supplemented with additional factors that encourage their attachment to a solid support including, but not limited to, type I, type II, and type IV collagen, concanavalin A, chondroitin sulfate, fibronectin, “superfibronectin” and/or fibronectin-like polymers, gelatin, laminin, poly-D and poly-L-lysine, Matrigel™, thrombospondin, and/or vitronectin. In certain embodiments, multi-well plates can be used (e.g., G-Rex culture plates).

In certain embodiments, the cell populations cultured according to the methods disclosed herein can be monitored to assess changes in the cells imparted by culturing (e.g., during a stage of culturing methods disclosed herein) to characterize the cell population produced. In some embodiments, the production of APS cells, DE cells, AFE cells, VPE cells, pharyngeal endoderm (PE) cells, third pharyngeal pouch endoderm (TPPE) cells, TEP cells, and/or TECs, including various sub-populations of TECs, can be assessed by determining expression of markers characteristic of these cell populations. In some embodiments, DE cell-identifying markers can include protein markers and can include SOX17+ and/or FOXA2. In other embodiments, AFE cell-identifying protein markers expressed herein can include SOX2+, FOXA2+ and/or SOX 17−. In certain embodiments, VPE cell-identifying markers can include HOXA3+, HOXB1−, and/or NKX2.1−. In some embodiments, thymic (e.g., TEPs/TECs) cell-identifying markers can include SOX 17−, SOX2+ and/or FOXA2+; EPCAM+CD104+; EPCAM+CD205+, HLA-Class II+; or any combination thereof. In certain embodiments, thymic (e.g., TEPs/TECs) cell-identifying markers can include EPCAM+ CD104+, FOXN1, HLA-Class II+, KRT5+, and/or KRT8+ where more than about 5%, more than about 8%, more than about 10%, more than about 11%, more than about 12%, more than about 13%, more than about 14%, or more than about 15% of the cells can be identified as EPCAM+ CD104+ KRT5+ and/or KRT8+. In some embodiments, thymic (e.g., TEPs/TECs) cell-identifying markers can include EPCAM+CD205+ wherein more than about 55%, more than about 56%, more than about 57%, more than about 58%, more than about 59%, more than about 60%, more than 61%, more than 62%, more than 63%, more than 64%, or more than 65% of the cells can be identified as EPCAM+CD104+. In some embodiments, thymic (e.g., TEPs/TECs) cell-identifying markers can include SOX 17−, SOX2+ and/or FOXA2+; EPCAM+ CD104+; CD205+, HLA-Class II+, FOXN1+, KRT5+, KRT8+; or any combination thereof wherein more than about 55%, more than about 56%, more than about 57%, more than about 58%, more than about 59%, more than about 60%, more than 61%, more than 62%, more than 63%, more than 64%, or more than 65% of the cells can be identified as SOX 17−, SOX2+ and/or FOXA2+; CD104+; EPCAM+ CD205+, HLA-Class II+, FOXN1+, KRT5+, KRT8+; or any combination thereof. In certain embodiments, thymic cells (e.g., TEPs/TECs) cell-identifying markers include HLA-Class II+ molecules, FOXN1+, KRT5+, KRT8+; or any combination thereof, wherein more than about 55%, more than about 56%, more than about 57%, more than about 58%, more than about 59%, more than about 60%, more than 61%, more than 62%, more than 63%, more than 64%, or more than 65% of the cells can be identified as expressing these protein markers. In accordance with these embodiments, expression of cell markers can be determined by detecting the presence or absence of the marker (e.g., detecting protein expression), and/or by analyzing expression of certain markers by measuring the level (e.g., detecting protein concentrations) at which the marker is present in the cells of the cell culture or cell population. Other methods known in the art can also be used to detect and/or quantitate marker gene expression. Non-limiting examples of methods suitable for use herein can include PCR, RT-PCR, qRT-PCR, immunoblotting, immunofluorescence, enzyme-linked immunosorbent assay (ELISA), flow cytometry, and the like.

In certain embodiments, methods disclosed herein provide for methods of preventing, reducing onset of, and/or treating one or more immune-mediated diseases or conditions in a subject by administering a composition including, but not limited to, thymic cells prepared by composition and methods disclosed herein. Non-limiting examples of such immune-mediated diseases or conditions include, but are not limited to, graft versus host disease (GvHD), DiGeorge syndrome, inflammatory bowel diseases (IBD), Crohn's disease Type-1 Diabetes, renal disease or renal condition or injury, psoriasis, asthma, allergies, rheumatoid arthritis, ankylosing spondylitis, cardiac conditions or cardiovascular disease, psoriasis, psoriatic arthritis, Behcet's disease, arthritis, viral infections (e.g., DNA viruses (Adenoviruses, Herpesviruses (e.g., Herpes simplex, type 1, Herpes simplex, type 2, Varicella-zoster virus, Epstein-barr virus, Human cytomegalovirus, Human herpesvirus, type 8), Papillomaviridae (e.g., Human papillomavirus), Poxviruses (e.g., Smallpox), Parvoviruses (e.g., Human bocavirus, Parvovirus B19), Hepadnaviridae (e.g., Hepatitis B virus) and/or Reoviruses (e.g., Rotavirus)), RNA viruses (Picornaviruses (e.g., coxsackievirus, hepatitis A virus, poliovirus, rhinovirus), Togaviruses (Rubella virus), Orthomyxoviruses (e.g., Influenza virus), and/or Rhabdoviruses (e.g., Rabies virus)), or reverse transcribing viruses (including, but not limited to, Retroviruses and Hepadnaviruses, Retroviridae (human immunodeficiency virus (HIV)), Metaviridae, Pseudoviridae, Caulimoviridae, I-Iepadnaviridae)). In some embodiments, the condition in need of treatment in a subject is GvHD, Type-1 diabetes, IBD, cardiovascular disease, renal injury, renal disease, the like. Also contemplated in the present disclosure are methods for treating one or more immune-mediated conditions that occur in certain types of cancers. Such cancers include, but are not limited to, carcinoma (e.g., breast, prostate, lung, pancreas, liver (e.g., hepatocarcinoma) or colon cancer), sarcoma (e.g., bone, cartilage, neuronal or fat (e.g., liposarcoma) cancers), lymphoma, leukemia (blood type cancers), blastomas (e.g., hepatoblastoma). In some embodiments, the condition in need of treatment in a subject can be restoration of immune function in a subject following for example, a thymectomy. In some embodiments, the condition in need of treatment in a subject can be restoration of immune function in a subject following a cancer treatment (e.g., chemotherapy, radiation therapy). In some embodiments, the condition in need of treatment in a subject can be restoration of immune function in a subject following one or more conditioning regimens as part of hematopoietic cell transplantation (HCT) (e.g., chemotherapy, radiation therapy). In some embodiments, the condition in need of treatment in a subject can be to reverse age-related thymic involution in the subject. In some embodiments, the condition in need of treatment in a subject can be to boost thymic function in the subject. In accordance with these embodiments, a boosted thymic function such as supplementation and/or replacement therapy in the subject can be used as a boost to a subject's response to vaccines, anti-cancer therapies and/or immunotherapy.

The term “subject” as used herein can refer to any mammal, including but not limited to, a non-human primate (for example, a monkey or great ape), livestock or pets such as a cow, a pig, a cat, a dog, a rat, a mouse, a horse, a goat, a rabbit, a sheep, a hamster, a guinea pig) or other subject. In some embodiments, the mammalian subject is a human such as an adult, a young child, adolescent, toddler, infant or fetus. In some embodiments, the mammalian subject is a human such as an older adult having an age of about 50 years or more, an age of about 60 years or more, an age of about 70 years or more, an age of about 80 years or more, an age of about 90 years or more, or an age of about 100 years or more.

In certain embodiments, the thymic epithelial progenitor (TEP) cells and/or thymic epithelial cells (TECs) produced using the methods disclosed herein can be used for generating functional thymic epithelium (TE) in a subject in need thereof. In certain embodiments, methods disclosed herein can include transplanting TEP cells generated according to the methods disclosed herein into a subject. In accordance with these embodiments, TEP cells generated according to the methods disclosed herein can generate TECs after transplantation into a subject. In some embodiments, at least about 5% of TEP cells generated according to the methods disclosed herein can differentiate into TEC cells after transplantation into a subject. In some embodiments, about 5% to about 99% of TEP cells generated according to the methods disclosed herein can differentiate into TECs after transplantation into a subject. In some embodiments, about 5%, about 10%, about 15%, about 20%, about 25%, about 30%, about 35%, about 40%, about 45%, about 50%, about out 55%, about 60%, about 65%, about 70%, about 75%, about 80%, about 85%, about 90%, about 95%, or about 99% of TEP cells generated according to the methods disclosed herein can differentiate into TECs after transplantation into a subject. In some embodiments, about 100% of TEP cells generated according to the methods disclosed herein can differentiate into TECs or TEs after transplantation into a subject.

In certain embodiments, methods disclosed herein can include transplanting one or more sub-populations of TECs generated according to compositions and methods disclosed herein into a subject. Examples of sub-populations of TECs generated according to the methods disclosed herein can include, but are not limited to, cortical thymic epithelial cell (cTEC) lineage cells, unipotent, bipotent and/or multipotent TEP cells, committed medullary thymic epithelial cell (mTEC) progenitors, immature mTECs, mature mTECs, post-AIRE mTECs, tuft cells, neuroendocrine cells, and/or myoid cells. In accordance with these embodiments, the one or more sub-populations of TECs generated according to the methods disclosed herein can generate TE after transplantation into a subject. In some embodiments, at least about 5% of TECs generated according to the methods disclosed herein can differentiate into TE after transplantation into a subject. In some embodiments, about 5% to about 99% of TECs generated according to the methods disclosed herein can differentiate into TE after transplantation into a subject. In some embodiments, about 5%, about 10%, about 15%, about 20%, about 25%, about 30%, about 35%, about 40%, about 45%, about 50%, about out 55%, about 60%, about 65%, about 70%, about 75%, about 80%, about 85%, about 90%, about 95%, or about 99% of TECs generated according to the methods disclosed herein can differentiate into TE after transplantation into a subject. In some embodiments, about 100% of TECs generated according to the methods disclosed herein can differentiate into TE cells after transplantation into a subject.

In certain embodiments, methods disclosed herein can include transplanting thymic cells produced generated according to the methods disclosed herein into a subject, wherein the thymic cells can have a mixed population of cell types. In some embodiments, thymic cells produced according to the methods disclosed herein to be transplanted into a subject can be a mixture of TEP, TEC and/or TE cells. In some embodiments, thymic cells produced according to the methods disclosed herein to be transplanted into a subject can be a mixture of TEP cells and one or more sub-populations of TECs. In some embodiments, thymic cells produced according to the methods disclosed herein to be transplanted into a subject can be a mixture of TEP cells and TECs, where about 1.0% to about 99.0% (e.g., about 1%, about 10%, about 20%, about 30%, about 40%, about 50%, about 60%, about 70%, about 80%, about 90%, about 99% to 100%) of the mixture are TEP cells. In some embodiments, thymic cells generated according to the methods disclosed herein to be transplanted into a subject can be a mixture of TEP cells and TECs, wherein about 1.0% to about 99.0% (e.g., about 1%, about 10%, about 20%, about 30%, about 40%, about 50%, about 60%, about 70%, about 80%, about 90%, about 99%) of the mixture are TECs.

In certain embodiments, methods disclosed herein can include transplanting thymic cells produced according to the methods disclosed herein into a subject in combination with one or more cells that are not thymic cells. In accordance with these embodiments, thymic cells generated according to the methods disclosed herein can be transplanted into a subject in combination with lymphatic endothelium cells, vascular endothelium cells, immune cells, mesenchymal cells, pericytes, red blood cells, or any combination thereof. In some embodiments, methods disclosed herein can include transplanting thymic cells generated according to the methods disclosed herein into a subject in combination with one or more cell-based immunotherapies. In some embodiments, methods disclosed herein can include transplanting thymic cells produced according to the methods disclosed herein into a subject in combination with one or more adoptive cell therapies. Non-limiting examples of adoptive cell therapies suitable for use herein can include Tumor-Infiltrating Lymphocyte (TIL) therapies, Engineered T Cell Receptor (TCR) therapies, Chimeric Antigen Receptor (CAR) T Cell therapies, Natural Killer (NK) Cell therapies, and the like.

For the purposes described herein, either autologous, allogeneic, or xenogeneic thymic cells prepared according to methods of the present disclosure can be administered to a subject. In accordance with these embodiments, the thymic cells can be either in undifferentiated, partially differentiated or fully differentiated forms, genetically altered or unaltered, introduced by direct injection to a tissue site, by infusion through a portal vein, in a bolus delivered to an organ, administered systemically, on or around the surface of an acceptable matrix, encapsulated or in combination with a pharmaceutically acceptable carrier. In some embodiments, pharmaceutical compositions disclosed herein can be formulated for parenteral administration, such as intravenous or intravascular, bolus infusion, intrarenal introduction, intracerebroventricular injection, intra-cisterna magna injection, intra-parenchymal injection, or any combination thereof. In some embodiments, pharmaceutical compositions disclosed herein can be formulated for intramuscular transplant and/or intramuscular injection. In some embodiments, pharmaceutical compositions disclosed herein can be formulated for orthotopic transplant.

In some embodiments, thymic cells produced by compositions and methods disclosed herein can be prepared for administering to a subject by any suitable method known in the art. In some embodiments, cells can be administered to a subject by localized or systemic injection. In some embodiments, thymic cell preparations can be administered by comparable methods to bone marrow implantation, such as through a renal artery or similar. In other embodiments, thymic cell preparations can be introduced directly to a site of interest such as an infection or other area in need of such a treatment. In other embodiments, thymic cell preparations disclosed herein can be introduced intramuscularly to the subject by transplant and/or injection. In yet other embodiments, in the process of transplanting at least one solid organ (e.g., kidney, lung, heart, liver or other) and/or a cellular transplant (e.g., bone marrow implantation) to a subject, thymic cells generated herein can be co-administered to a subject receiving such a transplant. In accordance with these embodiments, thymic cells generated by compositions and methods disclosed herein can be placed, for example, under a kidney capsule of a subject in need thereof for further implantation in the subject. Methods are known in the art for cellular implantation to improve integrity of transplanted cell populations, pharmaceutically acceptable excipients for delivery, and for improved impact of the cellular implantation thereof.

In some embodiments, the number of cells implanted into a subject can be a therapeutically effective number or amount. As used herein, a “therapeutically effective amount” can refer to the number of transplanted cells that have a treatment effect for a particular injury, disease or condition for which treatment is sought. For example, where the treatment is for tissue injury, implantation of a therapeutically effective number of cells can typically produce a reduction in the severity of the symptoms associated with the injury and in certain cases eliminate the injury. Persons or health professionals of skill in the art will understand how to determine proper cell dosages or concentrations of cell populations of use herein.

In some embodiments, the quantity thymic cells of the present disclosure to be administered can be optimized to achieve an optimal effect in a subject. Different scenarios can require optimization of the number of cells injected into a tissue of interest. For example, the quantity of cells to be administered can vary for the subject being treated. In some embodiments, between about 10⁴ to about 10¹⁰, or about 10⁶ to about 10⁸, or about 10⁹ or more thymic cells produced by compositions and methods disclosed herein can be administered in a single bolus or in multiple boluses for optimal effect. However, the precise determination of what would be considered an effective dose can be based on factors individual to each patient, including their size, age, degree of tissue injury/damage, and length of time from when the injury occurred. Dosages can be readily ascertained by those skilled in the art from this disclosure and the knowledge in the art and in consideration of transplantation doses or other cellular implant doses.

In some embodiments, thymic cells prepared according to the methods disclosed herein can be administered to a subject in need thereof alone, in combination with other therapeutic agents and/or treatments, and/or in combination with other transplanted organs and/or cells over the course of a day, for a few hours, daily, every other day, 2 times per week, weekly, every other week, monthly, or other appropriate treatment regimen. In accordance with these embodiments, thymic cells implanted by methods disclosed herein can include under a capsule of kidneys alone or in transplantation or implantation combinations. In addition, and further in accordance with the embodiments disclosed herein, a period of viability of the cells after administration to a subject can be a few hours (e.g., about 2 hours, about 6 hours, about 12 hours, about 24 hours), a few days (e.g., about 1 day, about 2 days, about 3 days, about 4 days about 5 days, about 6 days, about 7 days), weeks (e.g., about 2 weeks, about 4 weeks, about 6 weeks, about 12 weeks, about 40 weeks, about 52 weeks), to as long as several years (e.g., about 2 years, about 5 years), or even the life time of the subject, i.e., long-term engraftment.

In some embodiments, pharmaceutical formulations suitable for injection disclosed herein can include sterile aqueous solutions and dispersions or buffers for preserving cell populations of the instant inventions. A carrier or pharmaceutical excipient can be a dispersing medium containing, for example, water, saline, phosphate buffered saline, polyol (for example, glycerol, propylene glycol, liquid polyethylene glycol, and the like) and suitable mixtures thereof.

In some embodiments, certain additives which enhance the stability, sterility, and isotonicity of the thymic cell compositions, including antimicrobial preservatives, antioxidants, chelating agents, and buffers, can be added to the contemplated compositions herein. In some embodiments, antibacterial and antifungal agents can be added to reduce contamination of cultures for administration, for example, parabens, chlorobutanol, phenol, sorbic acid, and the like.

Sterile injectable solutions can be prepared by incorporating the thymic cells utilized in practicing the present disclosure in the required amount of the appropriate solvent with certain amounts of the other ingredients, as desired. Examples of compositions including the thymic cells of the present disclosure can include liquid preparations for administration, including suspensions. Such compositions can be in admixture with a suitable carrier, diluent, or excipient such as sterile water, physiological saline, glucose, dextrose, or the like.

Pharmaceutical compositions of the present disclosure can be provided as liquid preparations, e.g., isotonic aqueous solutions, suspensions, emulsions or viscous compositions, which can be buffered to a selected pH. The choice of suitable carriers and other additives can depend on the route of administration and the nature of the particular dosage form, e.g., liquid dosage form (e.g., whether the composition is to be formulated into a solution, a suspension, gel or another liquid form, such as a time release form or liquid-filled form). Solutions, suspensions and gels normally contain a major amount of water (e.g., purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose), can also be present. In some embodiments, pharmaceutical compositions contemplated herein can be isotonic, i.e., can have the same osmotic pressure as blood and lacrimal fluid. In other embodiments, agents can be provided to reduce cell lysing or other adverse effect on the cells for delivery to a subject.

In some embodiments, desired isotonicity of the cell compositions of the present disclosure can be accomplished using sodium chloride, or other pharmaceutically acceptable agents such as dextrose, boric acid, sodium tartrate, propylene glycol or other inorganic or organic solutes. Viscosity of the compositions, if desired, can be maintained at the selected level using a pharmaceutically acceptable thickening agent. Methylcellulose is readily and economically available and is easy to work with. Other suitable thickening agents include, for example, xanthan gum, carboxymethyl cellulose, hydroxypropyl cellulose, carbomer, and the like. The concentration of the thickener will depend upon the agent selected. The point is to use an amount, which will achieve the selected viscosity. Viscous compositions are normally prepared from solutions by the addition of such thickening agents.

A pharmaceutically acceptable preservative or cell stabilizer can be employed to increase the life of the compositions. If preservatives are used, it is well within the purview of the skilled artisan to select compositions that will not affect the viability or efficacy of the cells as described herein.

Pharmaceutical compositions of the present disclosure can be administered in dosages and by techniques well known to those skilled in the medical and veterinary arts taking into consideration such factors as the age, sex, weight, and condition of the particular patient, and the composition form used for administration (e.g., solid vs. liquid). Dosages for humans or other mammals can be determined without undue experimentation by the skilled artisan, from this disclosure, and the knowledge in the art.

In some embodiments, kits are contemplated of use to generate thymic cell populations disclosed herein or store or transport final and intermediary populations of cells for expansion or use. In certain embodiments, kits for use in treating or alleviating a targeted disease or condition treatable by use of thymic cells, such as an immune-mediated or immunocompromised condition or disease are disclosed herein. In some embodiments, the kit can include instructions for use in accordance with any of the methods described herein. Instructions found in a kit can include a description of administration of the thymic cell-containing composition, and optionally a second therapeutic agent, to treat, delay the onset, or alleviate a target disease as those described herein. The kit can further include a description of selecting a subject suitable for treatment based on identifying whether that individual has the target disease or condition, e.g., applying the diagnostic method as described herein and/or identifying symptoms in the subject. In some embodiments, the instructions can include a description for administering an antibody to a subject at risk of developing a disease or condition disclosed herein.

In some embodiments, instructions relating to the use of the thymic cell-containing can generally include information including but not limited to, dosage such as number of cells, dosing schedule, and route of administration for the intended treatment. Containers of kits can include unit dosing or bulk packages (e.g., multi-dose packages) or sub-unit doses. Kits can further include a delivery device such as a syringe, implant device or cellular delivery device. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions can be provided for practicing any of the methods described herein.

In some embodiments, kits can be in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle).

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

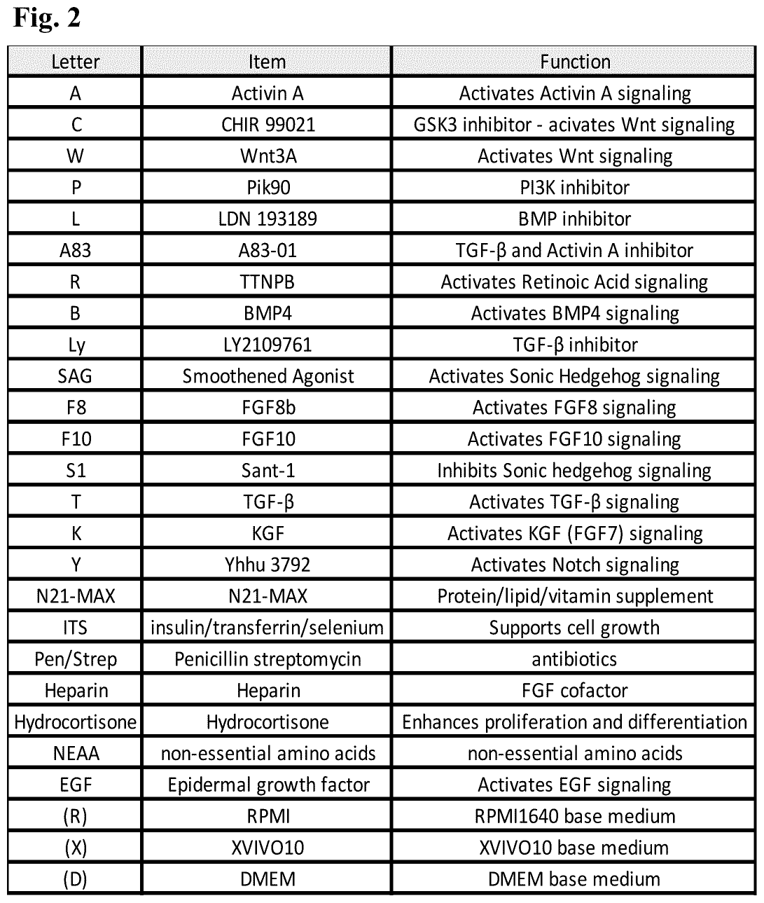

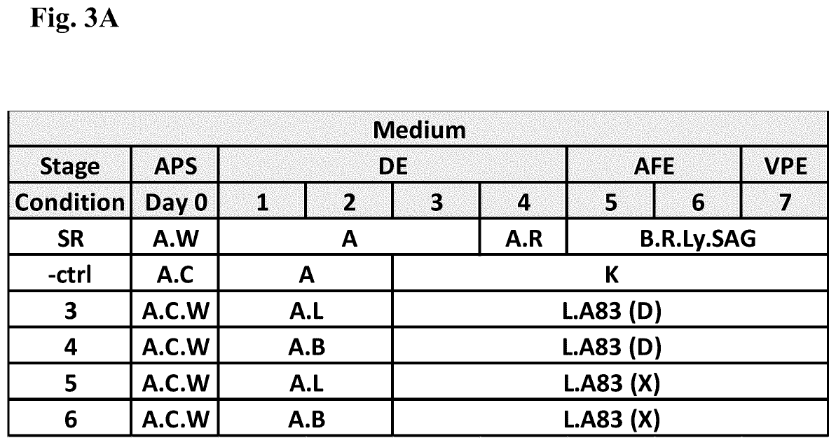

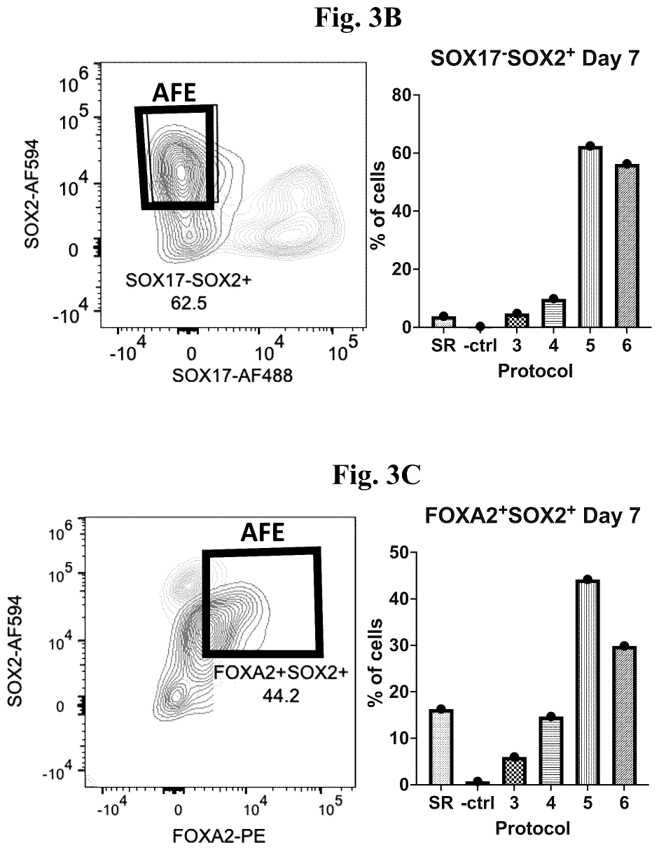

In one exemplary method, AFE induction from iPSCs was assessed. Specifically, six protocols for differentiation of iPSCs were tested to assess the effects of: optimized anterior primitive streak induction (AW vs AC vs ACW); BMP4 signaling modulations during day 1 and 2 of direct differentiation on production of anterior foregut endoderm (AFE); and base medium. FIG. 3A is a schematic illustrating the timelines and culture conditions for the six protocols performed in the present exemplary method. FIG. 2 provides a chart of agents, modulators, additives, and base mediums used in this and other exemplary methods herein.

In this example, all six protocols followed the same volume schedule as illustrated in Table 1.

TABLE 1

| Day | −1 | 0 | 1 | 2 | 3 | 4+ |
|---|---|---|---|---|---|---|
| Volume (μL) | 500 | 300 | 350 | 450 | 600 | 750 |

SR condition. For condition “SR” (one of two control conditions), on Day 1, iPSC were plated for differentiation in mTeSR+ supplemented with ROCKi. In brief, iPSC were first dissociated with TrypLE as follows: a) Aspirated medium from each well to dissociate and washed with 1 mL of 1×PBS; b) Replaced with 1 mL of TrypLE and placed in incubator for 7 minutes. c) Gently taped plate to release iPSC, added 1 mL of mTeSR+ to each well, and pipetted gently to get to single cells; d) transferred to a 15 mL conical that contained 4 mL of mTeSR+ (total volume is now 5 mL); e) counted cells then pelleted. The pellet was resuspended, and cells were plated as follows: a) Resuspended iPSC at 10e6/mL in mTeSR+ supplemented with rock inhibitor; b) Plated 600,000 iPSC per well (60 μL of suspension) in a total of 0.5 mL of mTeSR+ with ROCKi; c) Gently tapped plate back and forth to distribute iPSC evenly and placed in incubator overnight.

On day 0, Anterior Primitive Streak (APS) specification in “medium AW” was performed as follows: a) Washed each well with 1×PBS; and b) Replaced medium (replaced composition) in each well with XVIVO10 (e.g. protein-rich media) supplemented with 1:5000 ITS, 100 ng/mL Activin A, and 50 ng/mL Wnt3a. On days 1-3, DL specification in “Medium A” was performed as follows: a) Washed each well with 1×PBS; b) Replaced medium in each well with XVIVO10 supplemented 1:2000 ITS and 100 ng/mL Activin A. On day 4, DE specification in “Medium AR” was performed as follows: a) Washed each well with 1×PBS; b) Replaced medium in each well with X-VIVO10 supplemented with 1:2000 ITS, 100 ng/mL Activin A, and 6 nM TTNPB. On days 5-6, AFE specification in “Medium BRLySAG” was performed as follows: a) Replaced medium in each well with X-VIVO10 supplemented with 20 ng/ml BMP4, 6 nm TTNPB, 5 pim Ly, and 100 ng/ml SAG.

Ctrl Condition. For condition “Ctrl” (one of two control conditions), on Day 1, iPSCs were plated for differentiation in mTeSR+ supplemented with ROCKi according to the method disclosed above. On day 0, Anterior Primitive Streak specification in “medium AC” was performed as follows: a) Washed each well with 1×PBS; and b) Replaced medium in each well with RPMI supplemented 0.2% N21, 1:5000 ITS, 100 ng/mL Activin A, and 2 μM CHIR. On days 1-3, DE specification in “Medium A” was performed as follows: a) Washed each well with 1×PBS; b) Replaced medium in each well with RPMI supplemented 0.2% N21, 1:2000 ITS, and 100 ng/mL Activin A. On days 3 and beyond, cells were cultured in “Medium K” wherein “Medium K” was RPMI supplemented 1% N21, 1:1000 ITS, and 50 ng/mL KGF.

Conditions 3-6. For all three exemplary experimental conditions, on Day 1, iPSC were plated for differentiation in mTeSR+ supplemented with ROCKi according to the method disclosed above. On day 0, Anterior Primitive Streak specification in “medium ACW” was performed for all three exemplary experimental conditions, as follows: a) Washed each well with 1×PBS; and b) Replaced medium in each well with RPMI supplemented 0.2% N21, 1:5000 ITS, 100 ng/mL Activin A, 2 μM CHIR, and 50 ng/mL Wnt3a. On days 1-2, DE specification in “Medium ALDN” or “Medium AB” was performed as follows: a) Washed each well with 1×PBS; b1) For conditions 3 and 5, replaced medium in each well with RPMI supplemented 0.2% N21, 1:2000 ITS, 100 ng/mL Activin A, and 250 nM LDN; or b2) For conditions 4 and 6, replaced medium in each well with RPMI supplemented 0.2% N21, 1:2000 ITS, 100 ng/mL Activin A, and 20 ng/mL BMP4. On days 3-4, DE specification in “Medium LDNA83” was performed as follows: a) Washed each well with 1×PBS; b1) For condition 3 and 4, replaced medium in each well with DMEM supplemented with 1% N21, 1:2000 ITS, 250 nM LDN, and 1 μM A83; or b2) For condition 5 and 6, replaced medium in each well with X-VIVO10 supplemented with 1:2000 ITS, 250 nM LDN, and 1 μM A83. On days 5-6, AFE specification in “Medium LDNA83” was performed as follows: a1) For condition 3 and 4, replaced medium in each well with DMEM supplemented with 1% N21, 1:2000 ITS, 250 nM LDN, and 1 μM A83; a2) For condition 5 and 6, replaced medium in each well with X-VIVO10 supplemented with 1:2000 ITS, 250 nM LDN, and 1 μM A83.

Cells from each condition were subjected to flow cytometry at day 7 to assess the amounts of resulting cell types by measuring marker expression. Endoderm derived cells were identified by FOXA2 expression. Anterior cells were identified by co expression of SOX2. If endodermal cells were further posterior, SOX17 co-expression was observed. AFE cells were marked as FOXA2+, SOX2+ and SOX17−. FIG. 3B illustrates a representative flow plot and quantification of SOX17⁻SOX2⁺ cells from each differentiation protocol. FIG. 3C illustrates a representative flow plot and quantification of FOXA2⁺SOX2⁺ cells from each differentiation protocol. The data demonstrated that the XVIVO10 base medium having high protein (conditions 5 and 6), enhanced production of SOX17⁻SOX2⁺FOXA2⁺ AFE compared to DMEM (Conditions 3 and 4). The data also demonstrated that SOX17-SOX2⁺FOXA2⁺ AFE generation was not influenced by BMP signaling modulation.

### Example 2

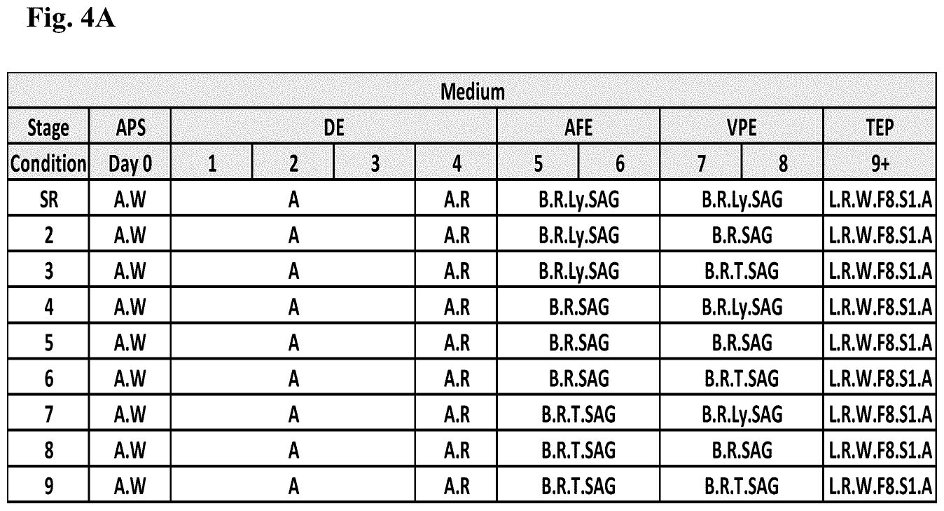

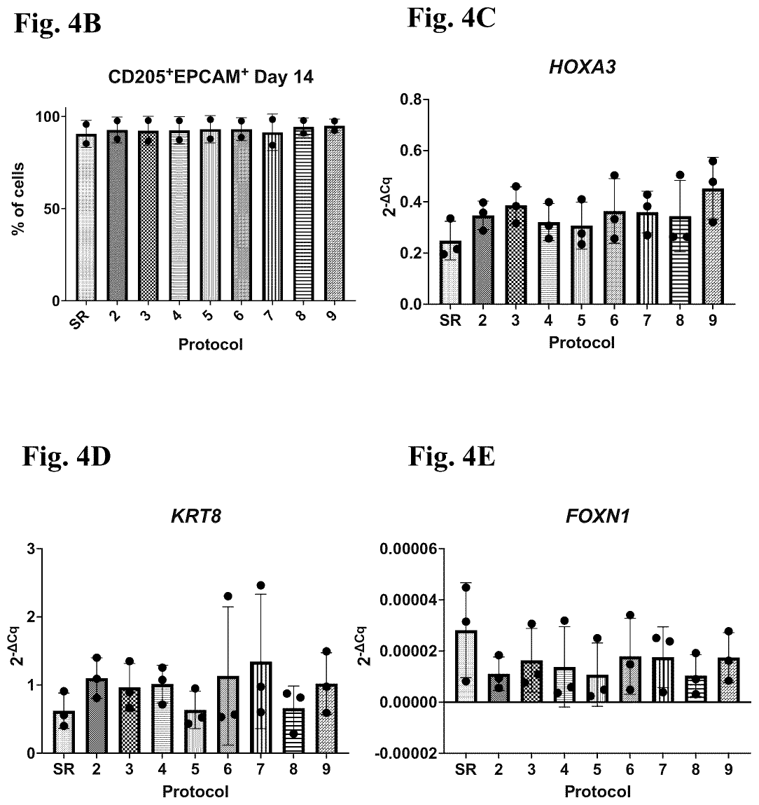

In another exemplary method, the influence of TGF-β signaling during generation of TEPs in two different stages was assessed. FIG. 4A is a schematic illustrating the timelines and culture conditions for the nine protocols performed in the present exemplary method. FIG. 2 provides a chart of agents, modulators, additives, and base mediums used in this and other exemplary methods herein.

All six protocols followed the same volume schedule as shown in Table 1. For all nine exemplary experimental conditions, on Day 1, iPSCs were plated for differentiation in mTeSR+ supplemented with ROCKi according to the method disclosed above. On day 0, Anterior Primitive Streak specification in “medium AW” was performed for all nine exemplary experimental conditions as follows: a) Washed each well with 1×PBS; and b) Replaced medium in each well with XVIVO10 supplemented 1:5000 ITS, 100 ng/mL Activin A, and 50 ng/mL Wnt3a. On days 1-3, DE specification in “Medium A” was performed for all nine exemplary experimental conditions as follows: a) Washed each well with 1×PBS; b) Replaced medium in each well with XVIVO10 supplemented 1:2000 ITS and 100 ng/mL Activin A. On day 4, DE specification in “Medium AR” was performed for all nine exemplary experimental conditions as follows: a) Washed each well with 1×PBS; b) Replaced medium in each well with XVIVO10 supplemented with 1:2000 ITS, 100 ng/mL Activin A, and 6 nM TTNPB.

On days 5-6, AFE specification was performed as follows: a) for conditions 1, 4, and 7, replaced medium in each well with XVIVO10 supplemented with 20 ng/ml BMP4, 6 nm TTNPB, 5 μm Ly, and 100 ng/ml SAG; b) for conditions 2, 5, and 8, replaced medium in each well with XVIVO10 supplemented with 20 ng/ml BMP4, 6 nm TTNPB, and 100 ng/ml SAG; c) for conditions 3, 6, and 9, replaced medium in each well with XVIVO10 supplemented with 20 ng/ml BMP4, 6 nm TTNPB, 1 ng/mL TGF-β, and 100 ng/ml SAG.

On days 7-8, VPE specification was performed as follows: a) for conditions 1-3, replaced medium in each well with XVIVO10 supplemented with 20 ng/ml BMP4, 6 nm TTNPB, 5 pin Ly, and 100 ng/ml SAG; b) for conditions 4-6, replaced medium in each well with XVIVO10 supplemented with 20 ng/ml BMP4, 6 nm TTNPB, and 100 ng/ml SAG; c) for conditions 7-9, replaced medium in each well with XVIVO10 supplemented with 20 ng/ml BMP4, 6 nm TTNPB, 1 ng/mL TGF-3, and 100 ng/ml SAG.

On day 9 and thereafter, TEP specification in “Medium LRWF8S1A” was performed for all nine exemplary experimental conditions as follows: a) replaced medium in each well with XVIVO10 supplemented with 1:2000 ITS, 250 nM LDN, 6 nm TTNPB, 50 ng/mL Wnt3a, 50 ng/mL FGF8, 250 nM SANT-1, 20 ng/mL Activin A, 10 μg/mL heparin, 500 ng/mL Hydrocortizone, 20 ng/mL EGF, and 1×Non-essential amino acids (NEAA).

The 9 protocols testing the effect of TGF-β inhibition (Ly), omission, or activation (T) on TEP induction were tested and analyzed by flow cytometry and qPCR on day 14. FIG. 4B illustrates flow plot quantification of CD205⁺EPCAM⁺ cells from each differentiation protocol and FIGS. 4C-4E illustrate a qPCR analysis of HOXA3, KRT8, and FOXN1 gene expression in the cells from each differentiation protocol.

During the AFE stage (days 5-6) the influence of: 1) inhibition of TGF-β signaling (Ly—conditions SR, 2, and 3); 2) no modulation of TGF-β signaling (conditions 4, 5, and 6); and 3) activation of TGF-β signaling (T—conditions 7, 8, and 9) was assessed. During the VPE stage (days 7-8) the influence of: 1) inhibition of TGF-β signaling (Ly—conditions SR, 4, and 7); 2) no modulation of TGF-β signaling (conditions 2, 5, and 8); and 3) activation of TGF-3 signaling (T—conditions 3, 6, and 9) was also assessed. Overall, the data showed no influence of TGF-3 signaling modulation on the expression of the TEP/TEC markers CD205 and EPCAM or the expression of the VPE marker I-LOXA3 or thymic epithelial progenitor cell (TEPs) markers KRT8 and FOXN1. These results unexpectantly showed that TGF inhibition was not required during AFE and VPE generation to efficiently establish thymic cells using direct differentiation which is in contrast to the currently practiced methods in the art for generation of thymic cells.

### Example 3

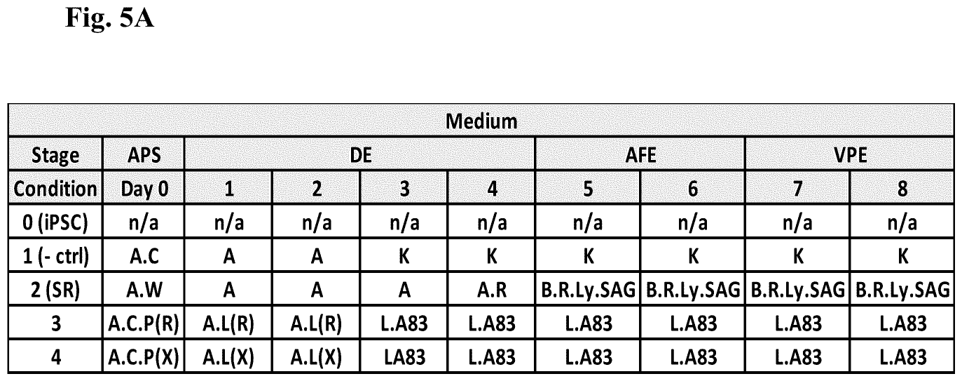

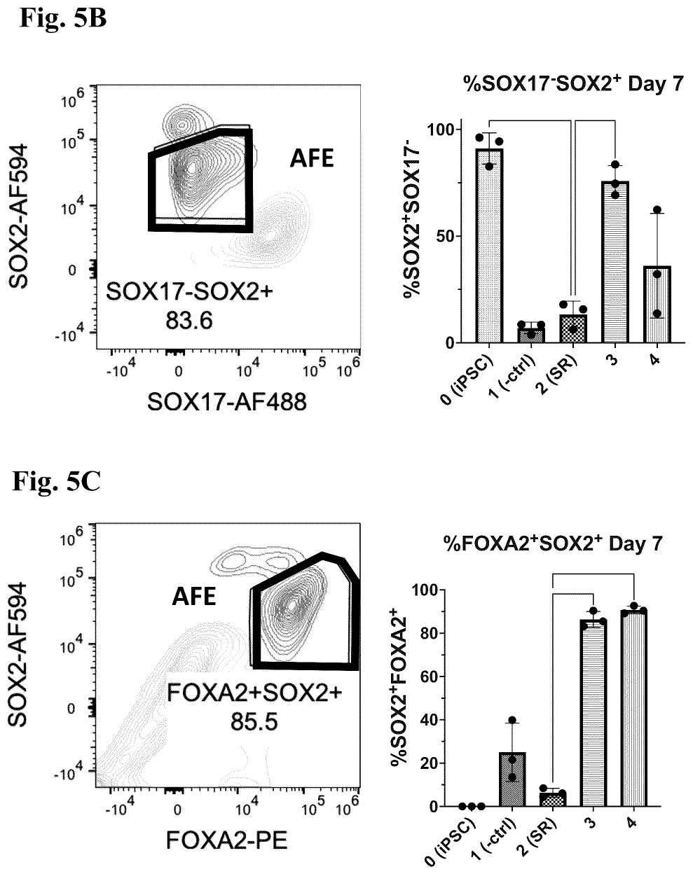

In another exemplary method, the effects of base media during the first stages of thymic cell differentiation were assessed. FIG. 5A is a schematic illustrating the timelines and culture conditions for the four protocols performed in the present exemplary method. FIG. 2 provides a chart of agents, modulators, additives, and base mediums used in this and other exemplary methods herein. All four protocols followed the same volume schedule as shown in Table 1. For all four exemplary experimental conditions, on Day 1, iPSCs were plated for differentiation in mTeSR+ supplemented with ROCKi according to the method disclosed above.

Condition SR. On day 0 of “SR”, Anterior Primitive Streak specification in “medium AW” was performed as follows: a) Washed each well with 1×PBS; and b) Replaced medium in each well with XVIVO10 supplemented 1:5000 ITS, 100 ng/mL Activin A, and 50 ng/mL Wnt3a. On days 1-3, DE specification in “Medium A” was performed as follows: a) Washed each well with 1×PBS; b) Replaced medium in each well with XVIVO10 supplemented 1:2000 ITS and 100 ng/mL Activin A. On day 4, DE specification in “Medium AR” was performed as follows: a) Washed each well with 1×PBS; b) Replaced medium in each well with X-VIVO10 supplemented with 1:2000 ITS, 100 ng/mL Activin A, and 6 nM TTNPB. On days 5-6, AFE specification in “Medium BRLySAG” was performed as follows: a) Replaced medium in each well with X-VIVO10 supplemented with 20 ng/ml BMP4, 6 nm TTNPB, 5 μm Ly, and 100 ng/ml SAG. On days 7-8, VPE specification in “Medium BRLySAG” was performed as follows: a) Replaced medium in each well with X-VIVO10 supplemented with 20 ng/ml BMP4, 6 nm TTNPB, 5 m Ly, and 100 ng/ml SAG.

Ctrl Condition. For condition “Ctrl” (one of two control conditions), on day 0, Anterior Primitive Streak specification in “medium AC” was performed as follows: a) Washed each well with 1×PBS; and b) Replaced medium in each well with RPMI supplemented 0.2% N21, 1:5000 ITS, 100 ng/mL Activin A, and 2 μM CHIR. On days 1-3, DE specification in “Medium A” was performed as follows: a) Washed each well with 1×PBS; b) Replaced medium in each well with RPMI supplemented 0.2% N21, 1:2000 ITS, and 100 ng/mL Activin A. On days 3 and beyond, cells were cultured in “Medium K” wherein “Medium K” was RPMI supplemented 1% N21, 1:1000 ITS, and 50 ng/mL KGF.

Conditions 3 and 4. For exemplary experimental conditions, on day 0 Anterior Primitive Streak specification in “medium ACP” was performed as follows: a) Washed each well with 1×PBS; and b1) for condition 3, replaced medium in each well with RPMI supplemented 0.2% N21, 1:5000 ITS, 100 ng/mL Activin A, 2 M CHIR, and 100 nM PIK-90; or b2) for condition 4, replaced medium in each well with XVIVO10 supplemented 1:5000 ITS, 100 ng/mL Activin A, 2 μM CHIR, and 100 nM PIK-90.

On days 1-2, DE specification in “Medium ALDN” was performed as follows: a) Washed each well with 1×PBS; b1) for condition 3, replaced medium in each well with RPMI supplemented 0.2% N21, 1:2000 ITS, 100 ng/mL Activin A, and 250 nM LDN; or b2) for condition 4, replaced medium in each well with XVIVO10 supplemented 1:2000 ITS, 100 ng/mL Activin A, and 250 nM LDN. On days 3-4, DE specification in “Medium LDNA83” was performed as follows: a) Washed each well with 1×PBS; b) replace medium in each well with X-VIVO10 supplemented with 1:2000 ITS, 250 nM LDN, and 1 μM A83 for both conditions 3 and 4. On days 5-6, AFE specification in “Medium LDNA83” was performed as follows: replaced medium in each well with X-VIVO10 supplemented with 1:2000 ITS, 250 nM LDN, and 1 μM A83 for both conditions 3 and 4.

The ability of 4 protocols to induce AFE were tested and analyzed via flow on day 7. FIG. 5B illustrates a representative flow plot and quantification of SOX17⁻SOX2⁺ cells from each differentiation protocol. FIG. 5C illustrates a representative flow plot and quantification of FOXA2⁺SOX2⁺ cells from each differentiation protocol. The data demonstrated that utilizing RPMI base medium (a very low protein medium compared to the high protein medium, XVIVO10) during early stages of thymic cell differentiation greatly enhanced generation of SOX17⁻SOX2⁺FOXA2⁺ AFE compared to XVIVO10. This data showed the that using the same signaling pathway modulators in conditions 3 or 4, both resulted in AFE marked by co-expression of FOXA2 and SOX2. However, only cells generated with condition 3 using RPMI as base media during days 0, 1, 2 do not co-express SOX17 compared to condition 4.

### Example 4

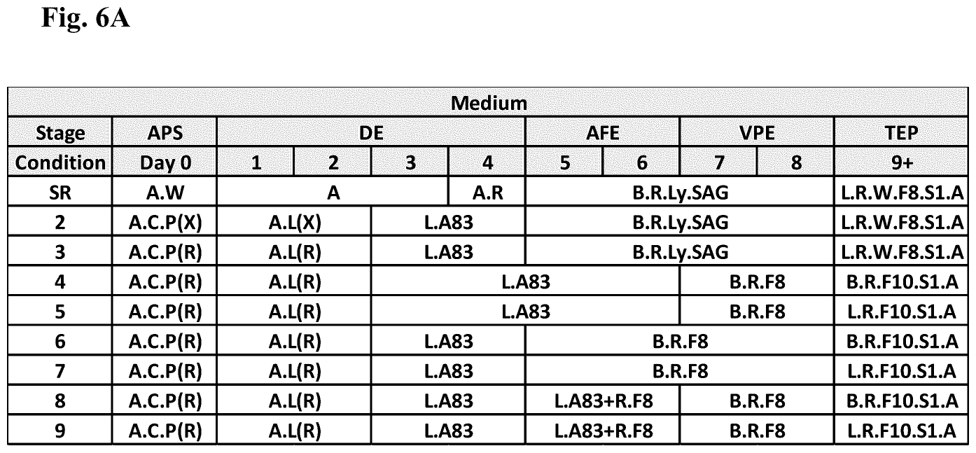

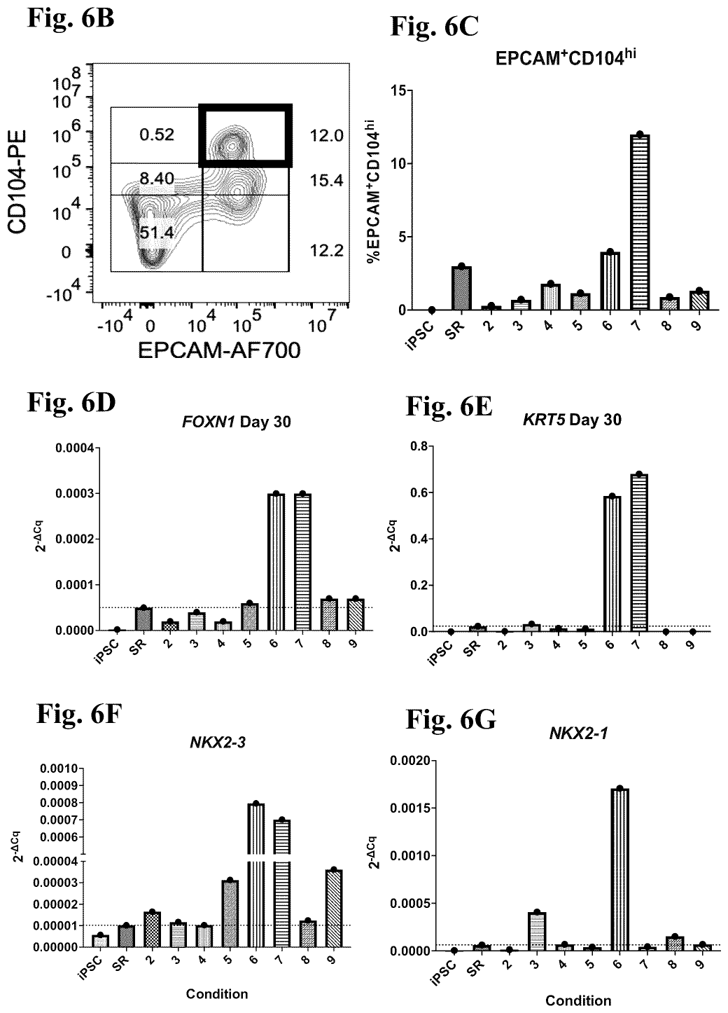

In another exemplary method, the timing of BMP signal modulation on the generation of TEPs/TECs was assessed. FIG. 6A is a schematic illustrating the timelines and culture conditions for the nine protocols performed in the present exemplary method, FIG. 2 provides a chart of agents, modulators, additives, and base mediums used in this and other exemplary methods herein. All nine protocols followed the same volume schedule as shown in Table 1. For all four exemplary experimental conditions, on Day 1, iPSCs were plated for differentiation in mTeSR+ supplemented with ROCKi according to the method disclosed above.

Condition SR. On day 0 of “SR”, Anterior Primitive Streak specification in “medium AW” was performed as follows: a) Washed each well with 1×PBS; and b) Replaced medium in each well with XVIVO10 supplemented 1:5000 ITS, 100 ng/mL Activin A, and 50 ng/mL Wnt3a. On days 1-3, DE specification in “Medium A” was performed as follows: a) Washed each well with 1×PBS; b) Replaced medium in each well with XVIVO10 supplemented 1:2000 ITS and 100 ng/mL Activin A. On day 4, DE specification in “Medium AR” was performed as follows: a) Washed each well with 1×PBS; b) Replaced medium in each well with X-VIVO10 supplemented with 1:2000 ITS, 100 ng/mL Activin A, and 6 nM TTNPB. On days 5-6, AFE specification in “Medium BRLySAG” was performed as follows: a) Replaced medium in each well with X-VIVO10 supplemented with 20 ng/ml BMP4, 6 nm TTNPB, 5 m Ly, and 100 ng/ml SAG. On days 7-8, VPE specification in “Medium BRLySAG” was performed as follows: a) Replaced medium in each well with X-VIVO10 supplemented with 20 ng/ml BMP4, 6 nm TTNPB, 5 μm Ly, and 100 ng/ml SAG. On days 7-8, VPE specification in “Medium BRLySAG” was performed as follows: replaced medium in each well with X-VIVO10 supplemented with 20 ng/ml BMP4, 6 nM TTNPB, 5 μm Ly, and 100 ng/ml SAG. On days 9 and thereafter, TPE specification in “Medium LRWF8S1A” was performed as follows: replaced medium in each well with X-VIVO10 supplemented with 1:2000 ITS, 250 nM LDN, 6 nM TTNPB, 50 ng/mL Wnt3a, 50 ng/mL FGF8, 250 nM SANT-1, 20 ng/mL Activin A, 10 μg/mL heparin, 500 ng/mL Hydrocortizone, 20 ng/mL EGF, and 1× non-essential amino acids (NEAA).

Conditions 2-9. For these exemplary conditions, on day 0, Anterior Primitive Streak specification in “medium ACP” was performed as follows: a) Washed each well with 1×PBS; and b1) for condition 2, replaced medium in each well with XVIVO10 supplemented with 1:5000 ITS, 100 ng/mL Activin A, 2 μM CHIR, and 100 nM PIK-90; b2) for conditions 3-9, replaced medium in each well with RPMI supplemented with 0.2% N21, 1:5000 ITS, 100 ng/mL Activin A, 2 μM CHIR, and 100 nM PIK-90. On days 1-2, DE specification in “Medium ALDN” was performed as follows: a) Washed each well with 1×PBS; and b1) for condition 2, replaced medium in each well with XVIVO10 supplemented with 1:2000 ITS, 100 ng/mL Activin A, and 250 nM LDN; b2) for conditions 3-9, replaced medium in each well with RPMI supplemented with 0.2% N21, 1:2000 ITS, 100 ng/mL Activin A, and 250 nM LDN. On days 3-4, DE specification in “Medium LDNA83” was performed as follows: a) Washed each well with 1×PBS; and b) Replaced medium in each well with X-VIVO10 supplemented with 1:2000 ITS, 250 nM LDN, and 1 μM A83. On days 5-6, AFE specification was performed as follows: a) For conditions 2 and 3, replaced medium in each well with X-VIVO10 supplemented with 20 ng/ml BMP4, 6 nM T TNPB, 5 μm Ly, and 100 ng/ml SAG; b) For conditions 4 and 5, replaced medium in each well with XVIVO10 supplemented with 1:2000 ITS, 250 nM LDN, and 1 μM A83; c) For conditions 6 and 7, replaced medium in each well with X-VIVO10 supplemented with 1:2000 ITS, 20 ng/ml BMP4, 6 μM TTNPB, and 50 ng/mL FGF8; For conditions 8 and 9, replaced medium in each well with X-VIVO10 supplemented with 250 nM LDN, 1 μM A83, 6 μM TTNPB, and 50 ng/mL FGF8. On days 7-8, AFE specification was performed as follows: a) For conditions 2 and 3, replaced medium in each well with X-VIVO10 supplemented with 20 ng/ml BMP4, 6 nM TTNPB, 5 μm Ly, and 100 ng/ml SAG; b) For conditions 4-9, replaced medium in each well with X-VIVO10 supplemented with 1:2000 ITS, 20 ng/ml BMP4, 6 μM TTNPB, and 50 ng/mL FGF8. On days 9 and thereafter, TEP specification was performed as follows: a) For conditions 2 and 3, replaced medium in each well with XVIVO10 supplemented with 1:2000 ITS, 250 nM LDN, 6 nM TTNPB, 50 ng/mL Wnt3a, 50 ng/mL FGF8, 250 nM SANT-1, 20 ng/mL Activin A, 10 μg/mL heparin, 500 ng/mL Hydrocortizone, 20 ng/mL EGF, and 1× Non-essential amino acids (NEAA); b) For conditions 4, 6, and 8, replaced medium in each well with X-VIVO10 supplemented with 1:2000 ITS, 20 ng/mL BMP4, 6 μM TTNPB, 2.5 ng/mL FGF10, 250 nM SANT-1, 20 ng/mL Activin A, 10 μg/mL heparin, 500 ng/mL Hydrocortizone, 20 ng/mL EGF, and 1× Non-essential amino acids (NEAA); c) For conditions 5, 7, and 9, replaced medium in each well with X-VIVO10 supplemented with 1:2000 ITS, 250 nM LDN, 6 μM TTNPB, 2.5 ng/mL FGF10, 250 nM SANT-1, 20 ng/mL Activin A, 10 μg/mL heparin, 500 ng/mL Hydrocortizone, 20 ng/m L EGF, and 1× Non-essential amino acids (NEAA).

Cells were analyzed via flow cytometry and qPCR on day 30. FIG. 6C illustrates a flow gating strategy and quantification of CD104^{hi}EPCAM⁺ cells for each protocol. FIGS. 6D-6G illustrates qPCR analysis of FOXN1, KRT5, NKX2-3 and NKX2-1 expression at day 30 for each protocol. The data showed how the timing of the introduction of BRF8 at day 7 (conditions 5 and 6), or day 5 (conditions 6 and 7), or the introduction of the nonredundant factors on top of LDN/A83 at day 5 (conditions 8 and 9) influenced TEP/TEC production. Data demonstrated that introduction of BRF8 at day 5 produced the TEPs/TECs with the highest EPCAM⁺CD104^{hi} populations and expressed the most cells with markers: FOXN1, KRT5, and NKX2-3.

The data also demonstrated how activation of BMP-4 signaling (conditions 4, 6, and 8) vs. inhibition of BMP-4 signaling (L—conditions 5, 6, and 9) in the TEP stage (Day 9+) influenced the generation of TEPs/TECs. Data demonstrated that inhibition of BMP-4 signaling gave the best on target gene expression in condition 7, specifically no NKX2-1 expression (a lung and thyroid marker) was seen in condition 7 while condition 6 expressed much more than any other condition Example 5

One of skill in the art understands that there are several causes of decline of the thymus and naïve T cells. For example, thymic atrophy (e.g., involution, immunosenescence), chemotherapeutic side effects, graft versus host disease (GvHD) and human immunodeficiency virus (HIV) can cause these declines. Development of a mature thymus results in distinct cortical and medullary regions. State of the art thymic epithelial cell differentiation protocols are deficient in functional TECs after long periods of time in vivo, low numbers of FOXN positive cells in vitro, low expression levels of TEP/TEC markers in vivo and in vitro providing a need for improved methods of generating these cells for therapeutic use and restoration. Compositions and methods disclosed herein provide for improved methods of producing functional thymic cells using novel supplemented medias, for example. In certain embodiments, early thymic progenitor signaling can enhance expression of FOXN1 during differentiation of thymic epithelial cells (TECs), for example CD40 ligands, RANK ligands and NOTCH Ligands. In some embodiments disclosed herein, it is demonstrated that FTP-derived signals (e.g., CD40L, RANKL, and NOTCH) can enhance iPSC-derived TEP expression of FOXN1 and MHC-II. In other embodiments, compositions and methods disclosed herein can be used to form stem cell-derived thymic organoids, produce some single positive T cell, and provide for further TEP/TEC maturation. In other embodiments, optimized TEP/TEC protocols disclosed herein can be combined with the stem cell-derived thymic organoid systems for improved outcome such as thymus function restoration.

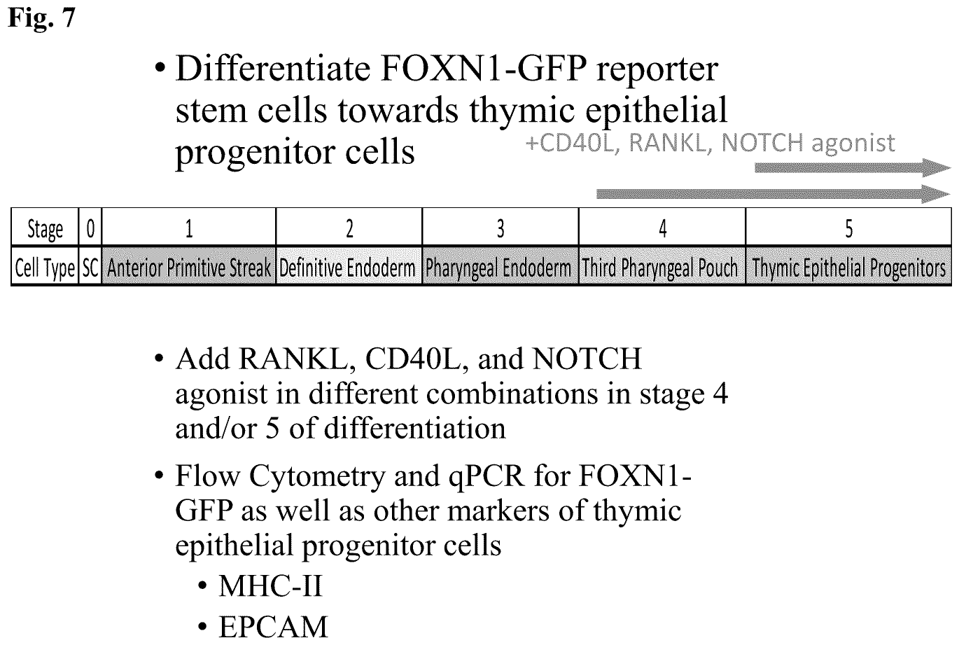

FIG. 7 illustrates an exemplary experimental set-up in accordance with certain embodiments of the present disclosure. This experimental set-up for production of add RANKL, CD40L, and NOTCH agonists in different combinations in stage 4 or 5 of differentiation of the progenitor cells. Flow cytometry and qPCR can be used to evaluate differentiation by assessing certain markers such as MHC-JJ and EPCAM markers which demonstrate effective differential using compositions and methods disclosed herein.

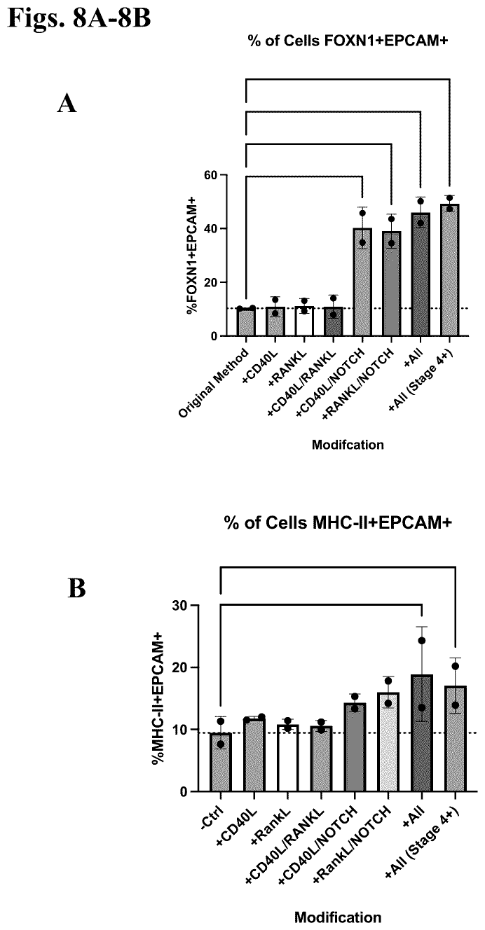

FIGS. 8A-8B illustrate additional stimulants used to enhance thymic cell production having improved marker representation, 8A and 8A illustrate percent representative marker outcome under the varying conditions in accordance with certain embodiments of the present disclosure. Previous results indicate production of about 10% FOXN1+EPCAM+ cells and addition of CD40L and/or RANKL by themselves in this study did not enhance production of these cells but addition of a NOTCH agonist enhanced FOXN1-GFP expression and also enhanced MIHC-II expression using compositions and methods disclosed herein. It was also observed that using all supplements positively enhanced expression of FOXN1+ and EPCAM+ cells (See FIGS. 8A and 8B).

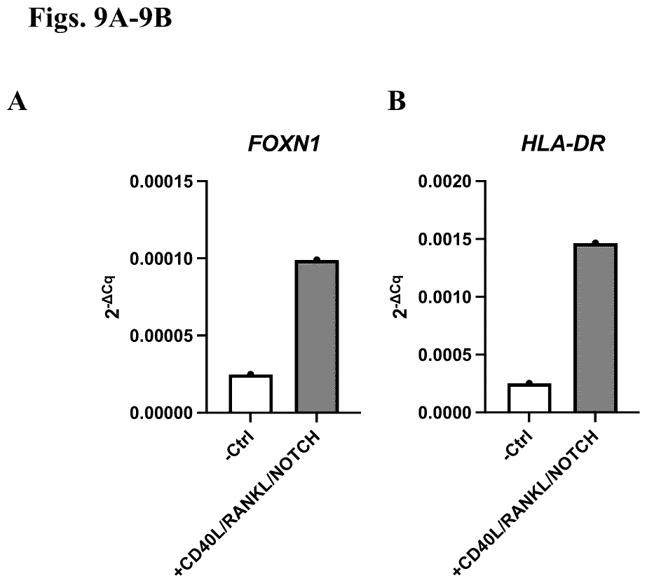

FIGS. 9A-9B illustrate exemplary results from qPCR detection of representative markers FOXN1 and MHC-II (e.g., HLA-DR) protein expression corroborated by gene expression under control and experimental conditions in accordance with certain embodiments of the present disclosure.

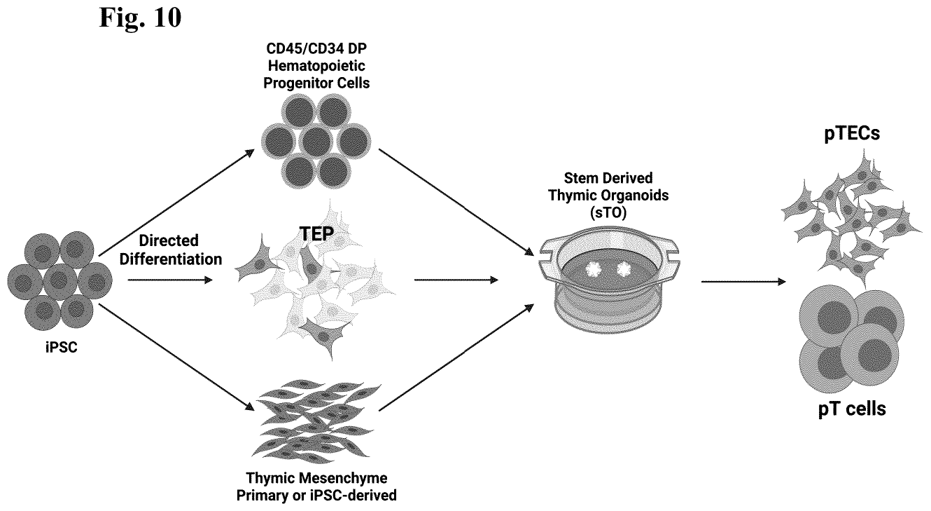

FIG. 10 is a schematic representation of a derivation of a stem cell-derived thymic organoid (sTO) in accordance with certain embodiments of the present disclosure.

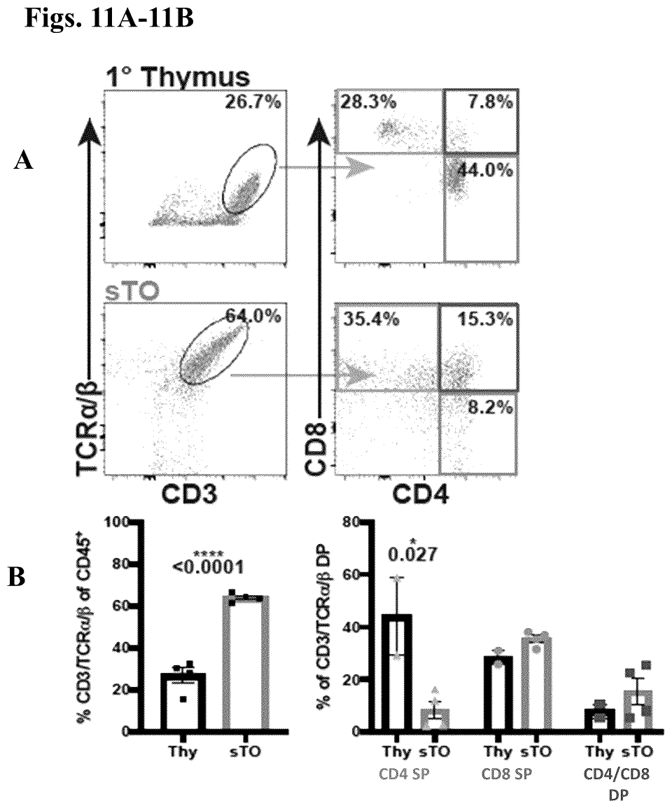

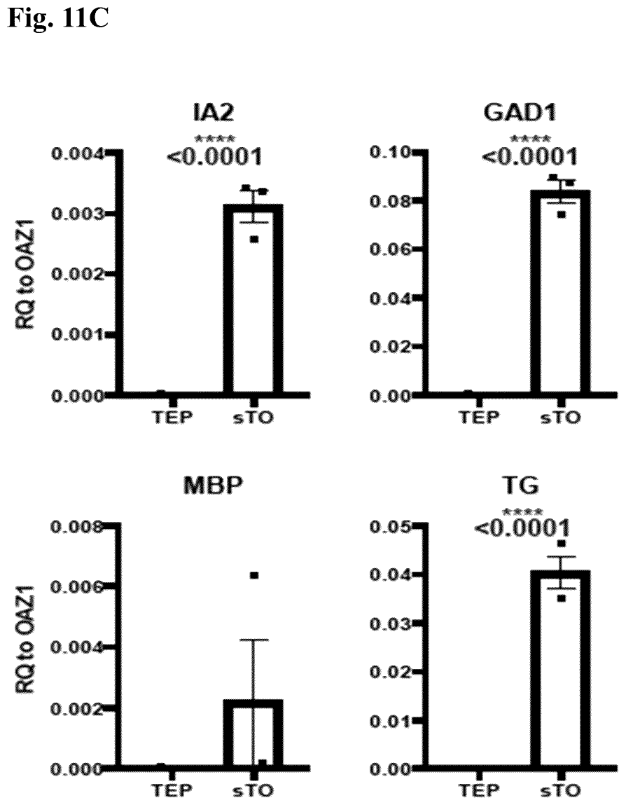

FIGS. 11A-11C illustrate exemplary results under various conditions of experimental thymic cell derivation protocols demonstrating where TEPS can further mature into sTOs using compositions and methods disclosed herein. 11A illustrates exemplary flow cytometry results; 11B illustrates production of sTOs compared to production of various T cell populations, and 11C represents assaying for tissue restricted antigen levels in TEP cells versus sTO under demonstrating superior expression of the tested antigens in the sTO populations (not shown are immunoassays illustrating expression of AIRE and AIRE plus DAPI, available upon request). As demonstrated herein, ETP-derived signals (CD40L, RAN KL, and NOTCH) were identified to enhance iPSC-derived TEP expression of FOXN1 and MHC-II. Stem cell-derived thymic organoids from further TEP/TEC maturation could be formed and single positive T cell populations produced.

All the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be provided to the COMPOSITIONS and METHODS and in the steps or in the sequence of steps of the MET HODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising: at least one bone morphogenetic protein (BMP) signaling activator and at least one fibroblast growth factor receptor 3 (FGFR3) activator and at least one Retinoic Acid (RA) signaling activator and a protein enriched cell culture medium wherein the protein concentration comprises about 0.5% (w/v) up to about 30.0% (w/v) protein and wherein the composition does not include a transforming growth factor-β (TGF-β) inhibitor.

2. The composition according to claim 1, further comprising definitive endoderm (DE) cells or anterior foregut endoderm (AFE) or ventral pharyngeal endoderm (VPE) cells.

3. The composition according to claim 1, wherein the BMP activator comprises BMP4 or SB4 or a combination thereof.

4. The composition according to claim 1, wherein the at least one FGFR3 activator comprises at least one of FGF8, FGF1, FGF2, FGF9 and Heparin.

5. The composition according to claim 1, wherein the at least one Retinoic Acid signaling activator comprises at least one of Retinoic acid, Vitamin A, TTNPB, AC261066, SR1078, SR221, BMS493, Fenretinide, AM580, Adapalene, and Ch55.

6. The composition according to claim 1, wherein the BMP activator comprises BMP4 or SB4 or a combination thereof; wherein the at least one FGFR3 activator comprises at least one of FGF8, FGF1, FGF2, FGF9 and Heparin; and wherein the at least one RA signaling activator comprises at least one of RA, Vitamin A, TTNPB, AC261066, SR1078, SR221, BMS493, Fenretinide, AM580, Adapalene, and Ch55.

7. A composition comprising: at least one FGF receptor 1 and FGF receptor 2 signaling activator; at least one RA signaling activator; at least one inhibitor of BM/IP signaling; at least one inhibitor of sonic hedgehog signaling and a protein enriched cell culture medium wherein the protein concentration comprises about 0.5% (w/v) to about 30.0% (w/v) protein and wherein the composition does not include an activator of BMP signaling.

8. The composition according to claim 7, further comprising at least one of an Wnt-signaling activator and an Activin A activator.

9. The composition according to claim 7, further comprising at least one of definitive endoderm (DE) cells, anterior foregut endoderm (AFE) cells, pharyngeal endoderm (PE) cells, ventral pharyngeal endoderm (VPE) cells, third pharyngeal pouch endoderm (TPPE) cells, and thymic cells.

10-14. (canceled)

15. A composition comprising: a protein enriched media having a protein concentration of about 0.5% to about 30.0% and at least one agent comprising at least one NOTCH pathway signaling activator, at least one CD40 pathway signaling activator, and at least one RANK signaling pathway activator and a combination thereof.

16. (canceled)

17. The composition according to claim 15, further comprising at least one population of cells comprising at least one of pharyngeal endoderm (PE) cells, third pharyngeal pouch endoderm (TPPE) cells and thymic epithelial progenitor (TEP) cells.

18. A method for differentiating mammalian pluripotent stem cells (PSCs) into anterior primitive streak cells and subsequently definitive endoderm (DE) cells, comprising incubating mammalian PSCs or anterior primitive streak cells or DE cells with a composition according to claim 1; and generating DE cells having capacity to differentiate into at least one of AFE, VPE and thymic cells.

19. The method according to claim 18, wherein incubating further comprises incubating in a low-protein or protein-free medium wherein low-protein comprises a concentration of less than 0.5% (w/v) in the medium for about 12 hours up to about 6 days prior to incubation in the composition according to claim 1.

20. The method according to claim 18, further comprising supplementing the composition or the medium with at least one of insulin, transferrin, selenium; insulin, transferrin, and selenium (ITS); insulin-transferrin-selenium-ethanolamine (ITS-X) from about day 0 up to about 24 hours of incubation at a dilution of about 1:50 to about 1:5000.

21-22. (canceled)

23. The method according to claim 18, further comprising incubating DE cells in a composition having the concentration of protein of at least 0.5% (w/v) protein up to about 30.0% (w/v) protein for about 12 hours up to about 7 days, or at least 5 days, or up to 7 days total having the at least one of a BMP inhibitor and the at least one of an Activin inhibitor to generate DE cells and the DE cells further capable of differentiating into at least one of anterior foregut endoderm (AFE) cells, ventral pharyngeal endodermal (VPE) and thymic cells.

24. The method according to claim 19, further comprising incubating DE or AFE cells as applicable in the composition having a concentration of protein of at least 0.5% (w/v) protein up to about 30% (w/v) protein about 12 hours up to about 6 days, or at least 5 days, or up to 6 days total having at least one of at least one FGF receptor 3 signaling activator, at least one Retinoic Acid signaling activator and at least one activator of BMP signaling activator to produce at least one of AFE and ventral pharyngeal endodermal (VPE) cells with the capacity to efficiently differentiate into thymic cells.

25. The method according to claim 24, further comprising after incubation for the about 12 hours up to about 6 days, or at least 5 days, or up to 6 days total, replacing the composition with a composition having a concentration of protein of about 0.5% (w/v) protein up to about 30.0% (w/v) protein further comprising at least one of at least one FGF receptor 1 and FGF receptor 2 signaling activator, at least one RA signaling activator, at least one inhibitor of BMP signaling, at least one inhibitor of sonic hedgehog signaling, at least one Activin A activator and at least one Wnt pathway signaling activator for about 12 hours up to about 60 days or more.

26. The method according to claim 25, wherein the final population of cells comprises thymic cells comprising at least one of TEP and TEC cells.

27. The method according to claim 18, further comprising replacing the composition with a composition comprising about 0.5% (w/v) to about 30% (w/v) protein containing at least one of: at least one FGF10 signaling activator, at least one RA signaling activator, at least one inhibitor of BMP signaling, at least one inhibitor of sonic hedgehog signaling and at least one Activin A activator and incubating the cells for about 12 hours up to about 60 days or more to generate thymic cells.

28. The method according to claim 18, wherein at least about 50% up to about 100% of the mammalian PSCs, the anterior primitive streak cells or the DE cells differentiate into AFE cells comprising SOX2+, FOXA2+ and SOX 17− and wherein at least about 40% up to about 100% of the mammalian AFE cells differentiate into VPE cells comprising HOXA3+HOXB1−NKX2.1−.

29-38. (canceled)

39. A kit comprising a composition according to claim 1; and at least one container.

40. A method for treating a health condition in a subject comprising administering a cell population generated by a method according to claim 18 to a subject in need thereof.

41. (canceled)
